(19) United States
(12) Patent Application Publication
(10) Pub. No.: US 2023/0039352 A1
(43) Pub. Date: Feb. 9, 2023

(54) CONDITIONALLY REPLICATING M. BOVIS BCG

(71) Applicant: Cornell University, Ithaca, NY (US)

(72) Inventors: Dirk Schnappinger, New York, NY (US); Sabine Ehrt, New York, NY (US)

(21) Appl. No.: 17/791,835

(22) PCT Filed: Jan. 8, 2021
(86) PCT No.: PCT/US2021/012671

§ 371 Date: Jul. 8, 2022

Related U.S. Application Data
(60) Provisional application No. 62/959,485, filed on Jan. 10, 2020.

(51) Int. Cl. A61K 39/04; C12N 1/20; C12N 15/74; C12N 15/63; A61P 31/06

(52) U.S. Cl. CPC A61K 39/04; C12N 1/20; C12N 15/74; C12N 15/635; A61P 31/06; C12N 2830/003; C12N 2800/101; A61K 2039/523

(57) ABSTRACT

Conditionally replicating recombinant cells, compositions and vaccines having the cells, and methods of using the cells, are provided.

### CROSS-REFENCE TO RELATED APPLICATIONS

This application claims the benefit of the filing date of U.S. application No. 62/959,485, filed Jan. 10, 2020, the disclosure of which is incorporated by reference herein.

### STATEMENT OF GOVERNMENT RIGHTS

This invention was made with government support under HHSN2612015000031 awarded by the Department of Health and Human Services and R01AI143788 awarded by the National Institute of Allergy and Infectious Disease. The government has certain rights in the invention.

### BACKGROUND

Tuberculosis (TB) is the most frequent cause of death from a single bacterial infectious agent despite the availability of a vaccine. TB causes more than a million deaths per year and it is estimated that over 2 billion people have been exposed to M. tuberculosis (Mtb). M. bovis Bacillus Calmette-Guerin (BCG) is the only TB vaccine in clinical use and has long been the most widely used vaccine worldwide (Anderson & Doherty, 2005). It also confers protection against leprosy. In addition, M. bovis BCG is used in immunotherapy of bladder cancer (Alexandroff et al., 1999). For example, after transurethral resection of bladder tumor (TURBT), BCG immunotherapy is delivered once per week for 6 weeks or up to 12 weeks. However, M. bovis BCG is a live vaccine and can cause illness as well as severe disease in immunocompromised humans (Casanova & Abel, 2002). For example, when BCG is administered to HIV infected individuals or when administered intravesically, e.g., to treat bladder cancer, it can cause disseminated disease (BCGiosis) (Perez-Jacoiste et al., 2014). If the infection spreads, it will lead to additional side effects including hepatitis, lung inflammation, or inflammation of the testes or prostate.

In much of the world, BCG is administered to infants and serious complications are rare, however, it has variable efficacy against pulmonary TB especially in adults. While BCG has a good safety record when administered by the traditional route (intradermal), it is not clear if it would have the same safety profile if administered by a different route.

### SUMMARY

The disclosure provides for a vector system and a recombinant host cell strain, for instance, bacterial strains, e.g., Mycobacterium strains including M. bovis BCG strains, modified with the vector system such that the strain depends on addition of an exogenous agent (e.g., TetOFF strains) for viability or growth, or that the strain is efficiently killed in the presence of the exogenous agent (e.g., TetON strains). The growth or viability regulating exogenous agents may be tetracyclines (e.g., doxycycline (doxy) or anhydrotetracycline (ATC)), other antibiotics including but not limited to macrolides (e.g., erythromycin) or pristinamycin, or other small molecules that can be used to specifically regulate the expression of bacterial genes, including but not limited to nitrile, cumate, benzoic acid derivatives (e.g., toluate), camphor, parabens, flavonoids (e.g., phloretin) or sugars (e.g., xylose or arabinose). Thus, the recombinant cells are growth-limited and/or growth-regulatable. The conditional control of growth or death of the modified strain is based on the use of a system that allows for temporal control of the expression of cytocidal, e.g., bacteriocidal, gene products (toxins). In one embodiment, the conditional control of growth or death of the modified strain is based on the use of one or more phage derived operons. In one embodiment, the conditional control of growth or death of the modified strain is based on regulation of the expression of bacteriocidal gene products, e.g., one or more bacteriocidal proteins such as a lysin, for instance, phage lysin, RNAse or DNase, or one or more bacteriocidal RNAs, such as small RNAs regulating the transcription or translation of growth essential genes, e.g., genes encoding RNA polymerase, inhA or other genes involved in fatty acid and/or mycolic acid biosynthesis, gyrase, ATP synthase, or the ribosome. In one embodiment, the conditional control is achieved by two phage-derived lysin operons, so-called kill-switches, whose expression is controlled by an exogenous agent, e.g., a tetracycline. The modified strains may be used as a vaccine, e.g., modified M. bovis BCG strains may be employed as a prophylactic or therapeutic, for example, for TB, or in therapies where growth or death of the modified strain provides a desirable result, e.g., modified M. bovis BCG strains may be used in immunotherapy, for example in the immunotherapy for cancer such as bladder cancer, or for non-cancer diseases.

In one embodiment, a vector is provided comprising at least two expression cassettes, wherein a first expression cassette comprises a first transcriptional regulatory region comprising a promoter operably linked to a first open reading frame encoding a first bacteriocidal lysin, and wherein a second expression cassette comprises a second transcriptional regulatory region comprising a promoter operably linked to a second open reading frame encoding a second bacteriocidal lysin, wherein expression from the first transcriptional regulatory region and the second transcriptional regulatory region is controlled in trans by at least one protein the activity of which is controlled by the exogenous agent, wherein the first and second open reading frames encode different lysins. In one embodiment, the protein that is controlled by the exogenous agent is a transcriptional regulator such as a tetracycline repressor (TetR) (e.g., revTetR, T38, TSC38, T10, T57 or TSC10) which recognizes the tet operator (tetO) which forms part of the transcriptional regulatory region. In one embodiment, TetR is constitutively expressed in a cell having the vector. In one embodiment, TetR is constitutively expressed in a cell before the cell is transformed with the vector. In one embodiment, TetR encoding nucleic acid is on the vector. In one embodiment, tetO is placed between nucleotides −10 and −35 in the promoter region that binds RNA polymerase in the transcriptional regulatory region. In one embodiment, tetO is placed between nucleotides 0 and −10 in the promoter region that binds RNA polymerase. In one embodiment, tetO is placed between nucleotides −35 and −45 in the promoter region that binds RNA polymerase. In one embodiment, the two expression cassettes are on a plasmid. In one embodiment, the plasmid is an integrating plasmid. In one embodiment, the plasmid is a non-integrating plasmid. As described herein, a TetON BCG strain (one where the presence of a tetracycline indirectly kills, e.g., lyses, the cell) was compared to WT BCG in vivo. The BCG TetON strain was eliminated faster than WT BCG (and thus was safer) and showed similar efficacy after reinfection with Mtb.

In one embodiment, a vector is provided comprising at least three expression cassettes, wherein a first expression cassette comprises a first transcriptional regulatory region comprising a promoter operably linked to a first open reading frame encoding a first bacteriocidal lysin, wherein a second expression cassette comprises a second transcriptional regulatory region comprising a promoter operably linked to a second open reading frame encoding a repressor protein, wherein a third expression cassette comprises a third transcriptional regulatory region comprising a promoter operably linked to a third open reading frame encoding a second bacteriocidal lysin, wherein expression from the first promoter is controlled by a first protein, the activity of which is controlled by an exogenous agent, wherein expression from the second promoter is controlled by a second protein, the activity of which is controlled by the exogenous agent, wherein the first and third open reading frames encode different lysins, and wherein the third transcriptional regulatory region binds the second protein (repressor) protein. In one embodiment, the repressor protein is any DNA binding protein that represses transcription, e.g., PipR, LacI, CRISPR/Cas, or AraC. As described herein, a TetOFF bacterial strain, e.g., one where the absence of tetracycline results in expression of one or more bacteriocidal gene products, induced cell death at a faster rate than a single toxin system and resulted in fewer escape mutants In one embodiment, TetON/OFF bacterial strains may be auxotrophic, e.g. unable to synthesize a cofactor such as biotin, which may further increase safety of the strain.

In one embodiment, the disclosure provides for isolated recombinant nucleic acid, e.g., DNA, comprising sequences for a dual toxin on/off switch that makes one or more genes, e.g., genes linked on a plasmid, constitutively active and constitutively turned off by administration of an exogenous agent (“on”) or active only in the presence of said agent (“off”). In one embodiment, the recombinant nucleic acid is part of a system, e.g., where nucleic acid encoding trans acting proteins are either linked to the switch elements or not linked to those elements but are otherwise present in the host cell genome or an autonomously replicating non-chromosomal element. In one embodiment, at least part of the system is integrated into the host cell chromosome. In one embodiment, at least part of the system is replicated autonomously from (not integrated into) the host cell chromosome. In one embodiment, the “dual-toxin on/off switch” employs sequences from two different lysin operons, e.g., that encode phage lysins. In one embodiment, the lysin operons are derived from phages that are specific for particular bacterial species, e.g., mycobacterial specific phage. In one embodiment, the system includes other sequences linked to the dual toxin on/off switch, e.g., a plasmid with other genes, for instance the proteins that act in trans to regulate expression of the lysins or desirable genes such as genes encoding immunostimulatory gene products. In one embodiment, the system is introduced to a host cell, such as a BCG strain. Also provided is a method to control gene expression using a dual toxin on/off switch and a separate (exogenous) agent that activates the switch, e.g., a tetracycline, including but not limited to doxycycline or anhydrotetracycline. In one embodiment, the exogenous agent is added to the host cell to turn on the expression of the cytocidal (toxin) genes, e.g., the toxin genes are not expressed in the absence of the exogenous agent. In one embodiment, the presence of the exogenous agent suppresses expression of the toxin genes and the withdrawal of the exogenous agent allows for toxin expression.

Further provided is a method to vaccinate against TB or treat TB. In one embodiment, a composition comprising a host cell having the system (recombinant host cell) is administered to a mammal, e g , a human, in an amount effective to induce a protective immune response to Mycobacterium tuberculosis (Mtb) infection. In one embodiment, the composition is administered and at a subsequent time, e.g., one week, one month, two months or more, an exogenous agent is administered that results in expression of bacteriocidal gene products (the dual toxins), thereby eliminating the host cell from the mammal. In one embodiment, a composition comprising a host cell having the system is administered to a mammal, e g , a human, in an amount effective to induce a therapeutic immune response to Mycobacterium tuberculosis infection. In one embodiment, the composition is administered and an exogenous agent is administered that suppresses expression of bacteriocidal gene products (the dual toxins), e.g., until a protective immune response is elicited, for instance, the agent is administered for at least one week or one to two months, then the administration of the exogenous agent is withdrawn, resulting in the dual toxin expression, thereby eliminating the host cell from the mammal In one embodiment, the mammal is an adult, e.g., a human over the age of 17. In one embodiment, the mammal is an infant or newborn, e.g., a human that is up to 3 months old. In one embodiment, the mammal is a youth (juvenile/adolescent), e.g., a human that is up to 17 years old. In one embodiment, administration protects or inhibits TB disease progression. In one embodiment, the administration protects or inhibits Mtb reinfection. In one embodiment, a series of doses is administered, e.g., a primary immunization dose and one to two boosters. In one embodiment the mammal is also administered an anti-mycobacterial drug, e.g., isoniazid, rifamycin, ethambutol, pyrazinamide, fluoroquinolone, amikacin, capreomycin, bedaquiline, linezolid, pretomanid, or any combination thereof. In one embodiment, the mammal is infected with a drug-resistant strain of M. tuberculosis. In one embodiment, a dose of the recombinant cell comprises anywhere from 10e1 to 10e8 CFU. In one embodiment, a dose of the recombinant cell comprises anywhere from 10e1 to 10e4 CFU. In one embodiment, a dose of the recombinant cell comprises anywhere from 10e5 to 10e8 CFU. In one embodiment, a dose of the recombinant cell comprises anywhere from 10e2 to 10e5 CFU.

Also provided is a method to inhibit or treat bladder cancer. In one embodiment, a composition comprising a host cell having the system is administered to a mammal, e g , a human, in an amount effective to inhibit or treat the cancer. In one embodiment, the host cell is propagated in vitro, e.g., in the presence of an exogenous agent, and then the composition is administered. In one embodiment, the composition is administered and optionally at a subsequent time, e.g., one week, one month, two months or more, an exogenous agent is administered that results in expression of cytocidal gene products such as bacteriocidal gene products (the dual toxins), thereby eliminating the host cell from the mammal In one embodiment, the composition is administered and an exogenous agent is administered that suppresses expression of cytocidal gene product bacteriocidal gene products (the dual toxins), e.g., until cancer treatment is terminated. In one embodiment, the agent is administered for at least one week and up to six months or a year or more, then the administration of the exogenous agent is withdrawn, resulting in the dual toxin expression, thereby eliminating the host cell from the mammal.

The modified (recombinant) cells described herein may be administered to a mammal, such as a human, by any route, e.g., intravenously, and are of value in numerous applications, e.g., cancer immunotherapy and in preventing or treating tuberculosis. In one embodiment, multiple doses of a composition comprising the modified cells may be administered.

In one embodiment, the cell modified with the vector is BCG Tokyo, Moreau, Russia or Sweden, or another isolate that secretes MPB70, has two copies of the insertion sequence IS6110, or contains methoxymycolate and MPB64 genes, or any combination thereof. In one embodiment, the cell modified with the vector is BCG Pasteur, Copenhagen, Glaxo or Tice, or an isolate that secretes very little MPB70, has a single copy of the insertion sequence IS6110, or does not contain the methoxymycolate and MPB64 genes, or any combination thereof.

In one embodiment, the cell modified with the vector is a BCG strain producing large amounts of autologous protective antigens, e.g., the 30-kDa, major secreted protein of M. tuberculosis, a strain that has lost ESAT—from region RD1 of BCG, a strain expressing a heterologous gene that facilitates presentation of antigens by MHC class I molecules, astrain expressing one or more cytokines, including IL-2, IFN-γ or others, in an attempt to enhance the immuno-stimulatory properties of BCG, or a strain that expresses antigens of other pathogens, e.g., antigens of a virus, a bacterium that is not Mycobacteria or a parasite, or a strain that has little to no expression of immunodominant antigens of M. bovis, e.g., due to deletion or selection, immunodominant antigens including but not limited to ESAT-6, CFP10, Ag85, MPB64, MPB70, or MPB83).

In one embodiment, the cell modified with the vector is an attenuated strain of M. tuberculosis.

The disclosure thus provides methods for producing recombinant cells for preventing or treating tuberculosis in humans and animals, as well as preventing or treating leprosy, preventing or treating other mycobacterial diseases or preventing or treating other intracellular pathogen infection, and cancer. In one embodiment, the recombinant cell is a biotin or lipoic acid auxotroph. In one embodiment, the biotin auxotroph comprises nucleic acid encoding a biotin dependent protein, e.g., a biotin dependent enzyme, and/or a biotin-protein ligase. In one embodiment, the lipoic acid auxotroph comprises nucleic acid encoding a lipoic acid liase acceptor protein, and/or a lipoic acid-protein ligase.

### BRIEF DESCRIPTION OF FIGURES

FIGS. 1A-1D. Tet-killed (TetON) and Tet-addicted (TetOFF) M. bovis BCG strains. A) Exemplary design of the kill-switches in a TetON configuration. Two lysin operons are expressed under control of a wild-type TetR. The addition of ATC/doxy induce expression of both lysin cassettes. B) Growth of the TetON strain on agar plates with and without ATC. When added, ATC was supplied via a paper disc placed in the center of the agar plate. C) Exemplary design of the kill-switches in a TetOFF configuration. One lysin operon is expressed under control of a reverse TetR (Klotzsche et al., 2009). The second lysin operon is expressed under control of the PipTetOFF system (Boldrin et al., 2010). ATC/doxy represses expression of both lysin cassettes. D) Growth of single/dual-lysin strains with and without ATC. When added, ATC was supplied via a paper disc placed in the center of the agar plate.

FIG. 2. Constructing a TetON BCG::D29-L5 lysin strain. Exemplary TetON system using TetR tsc10 to regulate two different expression cassettes expressing different phage lysins. In the absence of a tetracycline, ATC in this example, the lysins are not expressed. In the presence of the tetracycline, both lysins are expressed.

FIG. 3. Growth of a TetON BCG::D29-L5 lysin strain with and without ATC on agar plates. WT and recombinant BCG with expression cassettes for two different phage lysins were cultured on plates with and without a tetracycline. The plates on the right show that the recombinant strain is lysed in the presence of the tetracycline.

FIGS. 4A-4B. Growth of various BCG TetON strains with different concentrations of ATC. A) Growth of WT and a recombinant BCG TetON strain in the presence of increasing doses of ATC. B) OD₅₈₀ measurements over time of cultures of WT and recombinant BCG with expression cassettes in the presence of varying amounts of a ATC

FIG. 5. Induction of lysin TetON kill switches at different stages of growth. OD580 measurements were used to monitor growth of a double lysin strain and WT after addition of 1 μg/mL ATC at different times after inoculation (D0+, indicates that ATC was added at inoculation, D2+ indicates that ATC was added two days after inoculation, D4+ indicates that ATC was added four days after inoculation, etc.). Decreasing OD580 values after addition of ATC indicate that the double lysin strain was lysed by ATC at all growth stages.

FIG. 6. Growth of WT and recombinant BCG with one or two lysin TetON kill switches in the presence or absence of ATC. Growth was measured as OD580 or CFU/mL in the presence or absence of 1 μg/mL ATC. The OD measurements demonstrate that both single-lysin strains as well as the double-lysin strain are strongly attenuated with ATC. The more sensitive CFU measurements reveal that the double-lysin strain gets killed faster and that the double lysin strain is less susceptible to phenotypic escape. Appearance of escape mutants can be seen by the growth that was detected for both single-lysin strains after day 4.

FIG. 7. Survival of and cytokine induction by a BCG TetON strain in macrophages. Macrophages were infected with WT and recombinant BCG with expression cassettes for two different phage lysins and compared to controls for growth (CFUs), and cytokine secretion. ATC induced death of the double lysin strain in macrophages and induced an altered cytokine profile relative to controls.

FIG. 8. Doxycycline sensitivity test of a BCG dual-lysin TetON strain. WT and recombinant BCG with two expression cassettes were exposed to different concentrations of doxycycline.

FIG. 9. High-dose doxycycline treatment induced death in WT BCG after mouse infection. Mice were injected with approximately 10e6 CFU WT BCG on day 0, then exposed to doxycycline (which was administered in the mouse chow, which contained 2,000 ppm doxy) on day 7. Spleens and lungs were collected on days 14, 28, 42 and 70 for CFU/organ analyses.

FIG. 10. Doxycycline accelerates elimination of a BCG double-lysin TetON strain compared to WT BCG. Mice were injected with approximately 10e6 CFU of recombinant BCG with two expression cassettes on day 0, then exposed to doxycycline on day 7. Doxycycline was administered in the mouse chow, which contained 2,000 ppm doxycycline. Spleens and lungs were collected on days 14, 28, 42 and 70 for CFU/organ analyses. That data shown in FIG. 9 have been added here to facilitate the comparison with WT BCG.

FIG. 11. WT BCG and a BCG dual-lysin TetON strain provide similar protection against Mtb in mice. Design of the experiment is shown in the top panel. Mice were injected with 10e6 CFU WT or recombinant BCG with two expression cassettes on day 0, then exposed to doxycycline on day 7. Spleen and lung were collected on days 14, 28, 56 and 84 for CFU/organ analyses. On day 90, mice were challenged with approximately 100 CFU of Mtb H37Rv by aerosol infection. Survival of the two BCG strains in lungs (left) and spleens (right) from day 0 to day 80 is shown in the middle panel.

FIG. 12. Double lysin BCG strain provides equal protection efficacy to WT BCG strain by i.v. route. Growth and survival of Mtb H37Rv were measured in mice vaccinated by WT BCG or a BCG dual-lysin TetON strain. Mtb CFUs were measured 7, 28 and 56 days after challenge. Results are plotted for each mouse (WT and recombinant BCG with two expression cassettes after challenge).

FIG. 13. General gating strategy.

FIG. 14. Multifunctional CD4 T cell-gating results (unvaccinated).

FIG. 15. Multifunctional CD4 T cell-gating results (vaccinated).

FIG. 16. Graph showing multifunctional CD4 T cells (expressing IFN-gamma, TNF-alpha and IL-2) increased upon vaccination with WT and double lysin BCG.

FIG. 17. Data showing that more activated CD4 and CD8 T cells were detected in vaccinated groups.

FIG. 18. Ag85 specific CD4 T cell increased in vaccinated groups.

FIG. 19. Lung parenchyma localized CD153+CD4+ T cell populations increased in vaccinated groups.

FIG. 20. Characteristics of CD153+CD4+ T cells. Most CD153+ T cells are activated effector memory T cells with expression of CD44^{hi}, CD62L^{lo}. Many CD153+ T cells express two (double positive) or three (triple positive) cytokines. PD1−, KLRG1+CD153+ T cells are increased at early timepoints post vaccination.

FIG. 21. TetON and TetOFF kill switches. Both systems express two different bacteriocidal gene products, e.g., proteins such as enzymes. For TetON, the presence of the tetracycline turns on expression of both gene products and thus kills the host strain. In contrast, in TetOFF, the tetracycline causes repression of the kill switches and provides for viability. TetOFF strains are thus addicted to tetracyclines for growth and survival.

FIG. 22. Growth of representative single lysin and dual lysin strain on agar plates with and without atc. The plates on the left contain no atc; in the plates on the right atc was applied via a paper disc to the center of the plate. For the single lysin strain spotted colonies grew all over both plates, with and without atc, but growth is most robust around the atc disc (where the atc concentration is highest). In contrast, the double lysin strain only grows in the area around the atc disc. Spotted colonies do not appear far away from the disc or on the plate without atc. Similar results were observed for other strains tested and demonstrate that double-lysine strains are more stable than single lysin strains as the colonies that grew far away from the ATC disc are escape mutants.

FIG. 23. Fraction of escape mutants in double lysin and single lysin BCG TetOFF strains. For this experiment bacteria that originated from single colonies were cultivated in liquid media with atc. CFUs were then enumerated on agar plates with or without ATC. The fraction of escape mutants was calculated by dividing the number of CFU on non-permissive media (without ATC) by the number of CFU on permissive agar plates (with ATC). These experiments confirm that double-lysine strains are less prone to phenotypic reversion than single-lysin strains.

FIG. 24. Growth of single lysin and double lysin BCG TetON strains is dose-responsive to the concentration of ATC and doxycycline.

FIG. 25. A double lysin BCG TetOFF strain induced faster bacterial death and is less prone to phenotypic reversion in the absence of tetracycline. Growth was measured as OD580 or CFU/mL in the presence or absence of 1 g/mL ATC. The OD measurements (left panel) demonstrate that both single-lysin strains as well as the double-lysin strain are strongly attenuated with ATC. However, the single-lysin mutants start to re-grow beginning day 11. This is due to growth of escape mutants. This was not observed for the double-lysin strain. The CFU measurements (right panel) reveal that the double-lysin strain gets killed faster and confirms that it is less susceptible to phenotypic escape.

FIG. 26. Growth and survival of single- and double-lysin TetOFF strains over time in the presence or absence of a tetracycline.

FIG. 27. Comparison of growth and survival of a BCG dual lysin TetOFF strain in C57BL/6 and SCID mice. C57BL/6 mice are immunocompetent whereas SCID mice are immunodeficient due to an absence of functional B and T cells. Both mice strains were infected with approximately 10e6 CFU of a BCG dual lysin TetOFF strain by tail vein injection. Growth and survival was monitored by plating for CFUs. In contrast to C57BL/6 mice, the immunocompromised SCID mice are unable to control an infection with BCG as is evident by the growth of the BCG dual lysin TetOFF strain in mice that received doxy. The CFU data demonstrate that the dual lysin strain can be eliminated from both immunocompetent and immunocompromised mice by removing doxy.

FIG. 28. Use of different toxins to control growth of mycobacteria. The three pictures on the left show that growth of mycobacteria, in this case Mycobacterium tuberculosis, can be regulated with various toxins (e.g. a DNase, an RNase, or protein that inhibits the essential gyrase). The graph on the right shows the fraction of escape for several toxin strains, two single lysin strains, a dual lysin strain and a dual toxin strain in which growth is regulated by one lysin and an RNase. As is evident from these data, dual toxin strains, e.g. the dual lysin strain or the lysin/RNase strain, are more stable than single toxin strains (e.g. single lysin strains) and have lower frequencies of escape.

FIG. 29. Exemplary sequences.

FIG. 30. Overexpression of biotin-protein ligase (BPL) and biotin-dependent enzymes extends growth of the biotin-auxotroph Mtb AbioB without extrabacterial biotin. WT Mtb and Mtb AbioB were grown with biotin, concentrated by centrifugation, washed and then transferred into biotin-free media. Growth was analyzed using measurements of optical density. Mtb AbioB stopped growing after about 11 days, whereas strains overexpressing BPL and the enzyme ACC5 continued to grow until at least day 18 post transfer.

FIG. 31. In vivo kill kinetics of BCG Tet OFF dual lysin strain in C57BL6 mice and in immune-compromised SCID mice (lack B and T cells).

FIG. 32. Vaccination experiment: CFU of vaccine strains: WT BCG and Tet OFF dual lysin BCG (i.v. vaccination).

FIG. 33. Vaccination induced immune response—multifunctional CD4 T cell analysis.

FIG. 34. Vaccination induced immune response—CD11α+CD4 T cell analysis.

FIG. 35. Vaccination induced immune response—CD153+CD4 T cell analysis.

FIG. 36. Vaccination induced immune response—CD44^{high} CD62L^{low} CD4 T cell analysis.

FIG. 37. Vaccination induced immune response-lung resident memory CD4 T cell analysis.

FIG. 38. Vaccination induced immune response—CD44^{high} CD62L^{low} CD8 T cells.

FIG. 39. Vaccination induced immune response—CD11α+CD8 T cell analysis.

FIG. 40. Vaccine efficacy of BCG Tet OFF dual strain compare to WT BCG.

FIG. 41. Fluctuation analysis of three different BCG Tet OFF dual lysin strains.

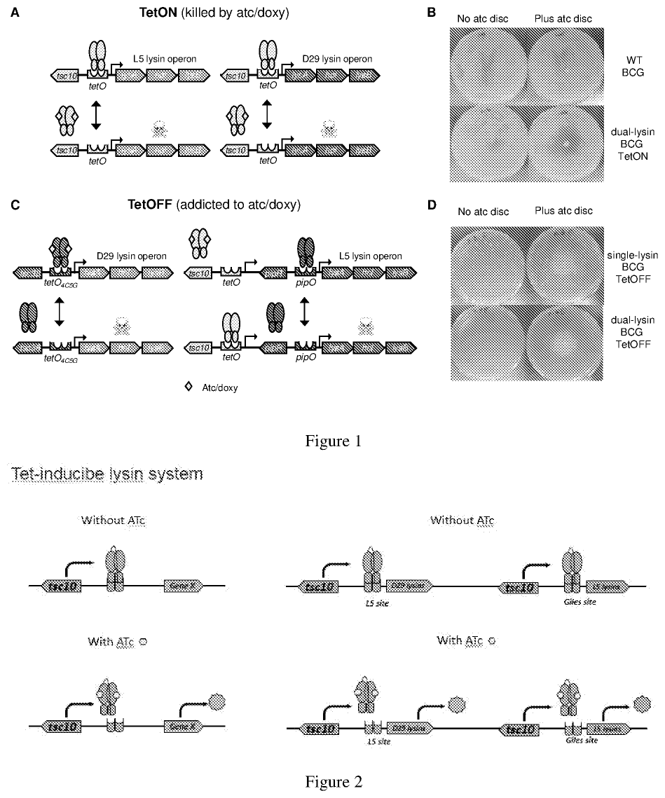

### DETAILED DESCRIPTION

Mycobacterium tuberculosis continues to be a leading cause of human deaths due to a single bacterial pathogen. Current efforts are focused on making better TB vaccines. The protective immunity against M. tuberculosis is mediated mainly by Th1-type CD4+ T cells and CD8+ T cells. These T cells secrete large amounts of cytokines, including gamma interferon (IFN-gamma) and tumor necrosis factor alpha (TNF-alpha, resulting in enhanced macrophage bactericidal activity and prevention of bacterial dissemination to the bloodstream and other tissues.

This disclosure relates to a recombinant cell, e.g., a recombinant BCG strain (rBCG). The recombinant cell is generated by incorporating a vector having at least two expression cassettes and optionally other genes, e.g., encoding mycobacterial antigens (e.g., Ag85B or CFP10) or immunostimulatory gene products such as human interleukin (IL) IL-12.

In one aspect, the disclosure relates to a recombinant bacterial cell strain, which comprises a vector having: a) a first expression cassette comprising an open reading frame for a bacteriocidal gene product operably linked to a controllable (via an exogenous agent) transcriptional regulatory region having a promoter effective for expression of the bacteriocidal gene product, wherein a trans-acting protein controls transcription therefrom; and b) a second expression cassette comprising an open reading frame for a second bacteriocidal gene product operably linked to a controllable (via an exogenous agent) transcriptional regulatory region having a promoter effective for expression of the bacteriocidal gene product, wherein a trans-acting protein controls transcription therefrom. In one aspect, transcription from each cassette is controlled by the same trans-acting protein. In one aspect, transcription from each cassette is controlled by a different trans-acting protein. Transcription may be controlled by a direct interaction between the trans-acting protein and the exogenous agent, and/or a direct interaction between the trans-acting protein and the transcriptional regulatory region. In one embodiment, at least one of the transcriptional regulatory regions comprises tetO.

In one aspect, the disclosure relates to a recombinant bacterial cell strain, which comprises a vector having: a) a first expression cassette comprising an open reading frame for a bacteriocidal gene product operably linked to a controllable transcriptional regulatory region having a promoter effective for expression of the bacteriocidal gene product, wherein a trans-acting protein controls transcription therefrom; b) a second expression cassette comprising an open reading frame for a second bacteriocidal gene product operably linked to a controllable transcriptional regulatory region having a promoter effective for expression of the bacteriocidal gene product, wherein a different trans-acting protein controls transcription therefrom; and c) a third expression cassette comprising an open reading frame for the different trans-acting protein operably linked to a controllable transcriptional regulatory region having a promoter effective for expression of the different trans-acting protein, wherein a third trans-acting protein controls transcription therefrom. Transcription may be controlled by a direct interaction between the trans-acting protein and the exogenous agent, and/or a direct interaction between the trans-acting protein and the transcriptional regulatory region. region. In one embodiment, at least one of the transcriptional regulatory regions comprises tetO.

In another aspect, the disclosure relates to a modified (recombinant) Bacille Calmette-Guerin (BCG) vaccine strain, which comprises: a) a first expression cassette comprising an open reading frame for a bacteriocidal gene product operably linked to a controllable transcriptional regulatory region having a promoter effective for expression of the bacteriocidal gene product, wherein a trans-acting protein controls transcription therefrom; and b) a second expression cassette comprising an open reading frame for a second bacteriocidal gene product operably linked to a controllable transcriptional regulatory region having a promoter effective for expression of the bacteriocidal gene product, wherein a trans-acting protein controls transcription therefrom. In one aspect, transcription from each cassette is controlled by the same trans-acting protein. In one aspect, transcription from each cassette is controlled by a different trans-acting protein.

In another aspect, the disclosure relates to a recombinant Bacille Calmette-Guerin (BCG) vaccine strain, which comprises: a) a first expression cassette comprising an open reading frame for a bacteriocidal gene product operably linked to a controllable transcriptional regulatory region having a promoter effective for expression of the bacteriocidal gene product, wherein a trans-acting protein controls transcription therefrom; b) a second expression cassette comprising an open reading frame for a second bacteriocidal gene product operably linked to a controllable transcriptional regulatory region having a promoter effective for expression of the bacteriocidal gene product, wherein a different trans-acting protein controls transcription therefrom; and c) a third expression cassette comprising an open reading frame for the different trans-acting protein operably linked to a controllable transcriptional regulatory region having a promoter effective for expression of the different trans-acting protein, wherein a third trans-acting protein controls transcription therefrom.

Further in another aspect, the disclosure relates to a method of inhibiting intracellular growth of Mycobacterium in a subject. The method comprises administering to a mammal the aforementioned recombinant cell strain in an amount effective for inhibition of Mycobacterium growth, e.g., in the lung cells of the mammal In one embodiment, the mammal has a Mycobacterium tuberculosis infection. In one embodiment, the recombinant cell may be a recombinant Mycobacterium cell. In one embodiment, the recombinant cell may be a recombinant Mycobacterium tuberculosis cell. In one embodiment, the recombinant cell may be a recombinant Mycobacterium bovis cell. In one embodiment, the recombinant cell may be a recombinant Bacille Calmette-Guerin (BCG).

The disclosure encompasses methods of inducing an immune response against, for example, M. tuberculosis, in a mammal. In some embodiments the immune response is a protective immune response, e.g., against M. tuberculosis. In some embodiments the methods comprise administering an effective dose of a pharmaceutical composition to a mammal in an amount that induces an immune response that is protective, e.g., against primary M. tuberculosis. The disclosure also encompasses methods of treating an infection, e.g., a M. tuberculosis infection, in a mammal. In some embodiments the methods comprise administering an effective dose of a pharmaceutical composition to a mammal so as to induce an immune response in the mammal that is protective, e.g., against an ongoing M. tuberculosis infection or reinfection.

The disclosure also encompasses methods of making a recombinant cell, e.g., a recombinant Mycobacterium such as a recombinant strain of M. bovis BCG. In some embodiments the methods comprise providing a vector comprising at least two expression cassettes, or at least three expression cassettes, as described herein, e.g., on the same vector into bacterial cells such as M. bovis BCG cells, and selecting cells, e.g., M. bovis BCG cells, that stably maintain the vector. In some embodiments the vector is an integrating vector.

### Definitions

As used herein, the term “overexpressing” or “increased expression” shall generally mean the expression of a specific protein or non-protein gene product from a vector in a cell or mammal is higher than that in a cell or mammal without the vector.

The term “vector” refers to genetic material that is used transfer linked genetic material to a target cell.

As used herein, the term “isolated” refers to a substance or entity that has been (1) separated from at least some of the components with which it was associated when initially produced (whether in nature or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

The term “peptide” a refers to a short polypeptide that contains at least 2 amino acids and typically contains less than about 50 amino acids and more typically less than about 30 amino acids. In some embodiments a peptide consists of from 2 to 50, from 2 to 20, from 2 to 10, from 5 to 10, from 5 to 15, from 5 to 20, from 10 to 20, from 10 to 30, from 10 to 40, from 10 to 50, from 20 to 40, or from 20 to 50 amino acids. The term as used herein encompasses analogs and mimetics that mimic structural and thus biological function.

The term “polypeptide” encompasses both naturally-occurring and non-naturally occurring proteins, and fragments, mutants, derivatives and analogs thereof. A polypeptide may be monomeric or polymeric. Further, a polypeptide may comprise a number of different domains each of which has one or more distinct activities. For the avoidance of doubt, a “polypeptide” may be any length greater two amino acids. Accordingly, a “polypeptide” may be a protein or a peptide.

The term “protein” refers to a polypeptide that comprises at least 50 amino acids. A “protein” may have the amino acid sequence of a naturally occurring protein or may be a modified derivative or a variant thereof.

As used herein, a protein has “homology” or is “homologous” to a second protein if the nucleic acid sequence that encodes the protein has a similar sequence to the nucleic acid sequence that encodes the second protein. Alternatively, a protein has homology to a second protein if the two proteins have similar amino acid sequences (Thus, the term “homologous proteins” is defined to mean that the two proteins have similar amino acid sequences). As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89.

The following six groups each contain amino acids that are conservative substitutions for one another: 1) Serine, Threonine; 2) Aspartic Acid, Glutamic Acid; 3) Asparagine, Glutamine; 4) Arginine, Lysine; 5) Isoleucine, Leucine, Methionine, Alanine, Valine, and 6) Phenylalanine, Tyrosine, Tryptophan. Sequence homology or similarity for polypeptides, which is also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof See, e.g., GCG Version 6.1.

An exemplary algorithm when comparing a particular polypeptide sequence to a database containing a large number of sequences from different organisms is the computer program BLAST (Altschul et al. (1990); Gish and States (1993); Madden et al. (1996); Altschul et al. (1997); Zhang and Madden (1997)), especially blastp or tblastn (Altschul et al. (1997)).

Exemplary parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62. The length of polypeptide sequences compared for homology will generally be at least about 16 amino acid residues, or at least about 20 residues, or at least about 24 residues, or at least about 28 residues, or more than about 35 residues. When searching a database containing sequences from a large number of different organisms, it may be useful to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990). For example, percent sequence identity between amino acid sequences can be determined using FASTA with its default parameters (a word size of 2 and the PAM250 scoring matrix), as provided in GCG Version 6.1, herein incorporated by reference.

In some embodiments, a variant has, for example, at least 85% overall sequence homology or similarity to its counterpart reference protein. In some embodiments, a variant has at least 90% overall sequence homology or similarity to the wild-type protein. In other embodiments, a variant has at least 95% sequence identity, or 98%, or 99%, or 99.5% or 99.9% overall sequence identity.

As used herein, “recombinant” may refer to a biomolecule, e.g., a gene or protein, or to a cell or an organism. The term “recombinant” may be used in reference to cloned DNA isolates, chemically synthesized polynucleotides, or polynucleotides that are biologically synthesized by heterologous systems, as well as proteins or polypeptides and/or RNAs encoded by such nucleic acids. A “recombinant” nucleic acid may be a nucleic acid linked to a nucleotide or polynucleotide to which it is not linked in nature. A “recombinant” protein or polypeptide may be (1) a protein or polypeptide linked to an amino acid or polypeptide to which it is not linked in nature; and/or (2) a protein or polypeptide made by transcription and/or translation of a recombinant nucleic acid. Thus, a protein synthesized by a microorganism is recombinant, for example, if it is synthesized from an mRNA synthesized from a recombinant nucleic acid present in the cell. A “recombinant” organism is an organism comprising a “recombinant” biomolecule. For example, a “recombinant” strain of M. bovis BCG is a strain of M. bovis BCG that comprises a “recombinant” nucleic acid.

The term “polynucleotide”, “nucleic acid molecule”, “nucleic acid”, or “nucleic acid sequence” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation. The nucleic acid (also referred to as polynucleotides) may include both sense and antisense strands of RNA, cDNA, genomic DNA, and synthetic forms and mixed polymers of the above. They may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. Other modifications can include, for example, analogs in which the ribose ring contains a bridging moiety or other structure such as the modifications found in “locked” nucleic acids.

The term “nucleic acid fragment” as used herein refers to a nucleic acid sequence that has a deletion, e.g., a 5′-terminal or 3′-terminal deletion compared to a full-length reference nucleotide sequence. In an embodiment, the nucleic acid fragment is a contiguous sequence in which the nucleotide sequence of the fragment is identical to the corresponding positions in the naturally-occurring sequence. In some embodiments, fragments are at least 10, 15, 20, or 25 nucleotides long, or at least 20, 30, 40, 50, 60, 70, 80, 90, 100, 110, 120, 130, 140, or 150 nucleotides long. In some embodiments a fragment of a nucleic acid sequence is a fragment of an open reading frame sequence. In some embodiments such a fragment encodes a polypeptide fragment (as defined herein) of the protein encoded by the open reading frame nucleotide sequence.

A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention.

The term “percent sequence identity” or “identical” in the context of nucleic acid sequences refers to the residues in the two sequences which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32, and even more typically at least about 36 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences. Pearson (1990). For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference. Alternatively, sequences can be compared using the computer program, BLAST (Altschul et al. (1990); Gish and States(1993); Madden et al. (1996); Altschul et al. (1997); Zhang and Madden (1997)), especially blastp or tblastn (Altschul et al. (1997)).

As used herein, an “expression control sequence” refers to polynucleotide sequences which affect the expression of coding sequences to which they are operatively linked. Expression control sequences are sequences which control the transcription, post-transcriptional events and translation of nucleic acid sequences. Expression control sequences include appropriate transcription initiation, termination, promoter sequences, operator sequences, enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to encompass, at a minimum, any component whose presence is essential for expression, and can also encompass an additional component whose presence is advantageous, for example, leader sequences and fusion partner sequences.

As used herein, “operatively linked” or “operably linked” expression control sequences refers to a linkage in which the expression control sequence is contiguous with the gene of interest to control the gene of interest, as well as expression control sequences that act in trans or at a distance to control the gene of interest.

As used herein, a “vector” is intended to refer to a nucleic acid molecule capable of transporting another nucleic acid to which it has been linked. One type of vector is a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain vectors are capable of directing the expression of genes to which they are operatively linked. Such vectors are referred to herein as “recombinant expression vectors” (or simply “expression vectors”).

The term “recombinant host cell” (or simply “recombinant cell” or “host cell”), as used herein, is intended to refer to a cell into which a recombinant nucleic acid such as a recombinant vector has been introduced. In some instances, the word “cell” is replaced by a name specifying a type of cell. For example, a “recombinant microorganism” is a recombinant host cell that is a microorganism host cell. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “recombinant host cell,” “recombinant cell,” and “host cell”, as used herein. A recombinant host cell may be an isolated cell or cell line grown in culture or may be a cell which resides in a living tissue or organism.

As used herein, the term “mammal” refers to any member of the taxonomic class mammalia, including placental mammals and marsupial mammals. Thus, “mammal” includes humans, primates, livestock, and laboratory mammals. Exemplary mammals include a rodent, a mouse, a rat, a rabbit, a dog, a cat, a sheep, a horse, a goat, a llama, cattle, a primate, a pig, and any other mammal In some embodiments, the mammal is at least one of a transgenic mammal, a genetically-engineered mammal, and a cloned mammal

### Exemplary Recombinant Strains

In one embodiment, the recombinant cell is a bacterial cell. In one embodiment the recombinant cell is a Mycobacterium. In one embodiment, the recombinant cell is a recombinant M. bovis. In one embodiment, the recombinant cell is a M. bovis BCG. The recombinant strains comprise a vector encoding one or more heterologous proteins including one or more bacteriocidal proteins and optionally one or more heterologous transcriptional regulatory proteins. In one embodiment, the bacteriocidal protein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% nucleic acid or amino acid sequence identity to any one of SEQ ID Nos. 20-29. In one embodiment, the transcriptional regulatory protein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% nucleic acid or amino acid sequence identity to any one of SEQ ID Nos. 1-7.

In some embodiments, the vector is linked to a chromosomal sequence in the recombinant cell. In some embodiments, the vector is autonomous (not linked) to a chromosomal sequence in the recombinant cell. In some embodiments, the transcriptional regulatory protein coding sequences are operably linked to endogenous transcriptional regulatory sequences. In some embodiments, the transcriptional regulatory protein coding sequences are operably linked to heterologous transcriptional regulatory sequences.

In some embodiments the vector sequences are on a plasmid. In some embodiments the vector sequence is integrated into a M. bovis BCG chromosome. In some embodiments the recombinant M. bovis BCG strain comprises a single copy of the vector sequence integrated on its chromosome. In some embodiments the recombinant M. bovis BCG strain comprises multiple copies of the vector sequence integrated on its chromosome. In one embodiment, the vector is a non-integrating vector.

The recombinant strains may be made by any suitable method known in the art. In some embodiments an integrating shuttle vector is electroporated into a cell and recombinant cells comprising the vector sequence, e.g., integrated into the host cell chromosome, are identified.

In some embodiments the use of the recombinant strain having the vector induces a protective immune response greater than the parent strain that lacks the vector when introduced into a mammal. In some embodiments, the virulence of the recombinant strain is equal to or lower than the virulence of the parent strain in a mammal. In some embodiments, the recombinant M. bovis BCG induces a protective immune response greater than the parent M. bovis BCG when introduced into a mammal. In another embodiment, the virulence of the recombinant M. bovis strain is equal to or lower than the virulence of the parent M. bovis BCG in the mammal. In some embodiments the mammal is a human

In some embodiments, the recombinant strain further comprises an antibiotic marker, e.g., on the vector. In some embodiments the antibiotic marker is removed from the recombinant strain after integration of the vector, so as the recombinant strain could be used for GMP production. In some embodiments an antibiotic marker is not introduced to the recombinant strain. In some embodiments the recombinant strain lacks an antibiotic marker.

### Methods of Making Recombinant Strains

The disclosure also encompasses methods of making a recombinant strain, e.g., a recombinant Mycobacterium strain such as a recombinant of M. bovis BCG. In some embodiments the methods comprise providing a vector comprising at least two expression cassettes as disclosed, introducing the vector into cells, and selecting cells that stably maintain the vector sequences. In some embodiments the vector is an integrating vector and the method may further comprise selecting cells in which the vector sequence has integrated into the host cell chromosome.

In one embodiment, M. bovis BCG cells used to make a recombinant strain of M. bovis BCG are from a commercially available BCG strain which has been approved for use in humans such as Pasteur, Frappier, Connaught (Toronto), Tice (Chicago), RIVM, Danish 1331, Glaxo-1077, Tokyo-172 (Japan), Evans, Prague, Russia, China, Sweden, Birkhaugh, Moreau and Phipps.

### Pharmaceutical Compositions

The disclosure encompasses compositions, particularly pharmaceutical compositions, comprising a recombinant cell, e.g., recombinant M. bovis BCG, and a pharmaceutically-acceptable carrier. The compositions may be, for example, for use for inducing a protective immune response against M. tuberculosis in a subject mammal and/or for use for treating M. tuberculosis infection in a subject mammal The compositions may be, for example, for use in inducing a protective immune response against leprosy and/or treating leprosy. The compositions may be, for example, for use in inhibiting or treating cancer.

In addition to the strains, these pharmaceutical compositions may contain suitable pharmaceutically-acceptable carriers comprising excipients and auxiliaries which facilitate processing of the living vaccine into preparations which can be used pharmaceutically. Further details on techniques for formulation and administration may be found in the latest edition of Remington's Pharmaceutical Sciences (Maack Publishing Co., Easton, Pa.). In some embodiments the pharmaceutical composition is suitable for intravenous, oral, subcutaneous, intradermal, inhalation or intravesicular administration.

The determination of the effective dose is well within the skill of the art worker. An effective dose refers to that amount of active ingredient, e.g., the number of cells administered, which in one embodiment induces an immune response against M. tuberculosis and/or ameliorates the symptoms of M. tuberculosis infection, and in another embodiment, is effective to inhibit or treat cancer, e.g., bladder cancer. Efficacy and toxicity may be determined by standard pharmaceutical procedures in experimental animals, e.g., ED50 (the dose effective in 50% of the population) and LD50 (the dose lethal to 50% of the population). The dose ratio of toxic to therapeutic effects is the therapeutic index, and it can be expressed as the ratio, LD50/ED50. Pharmaceutical compositions which exhibit large therapeutic indices amy be employed. Of course, ED50 is to be modulated according to the mammal to be treated or vaccinated. In this regard, the composition is suitable for human administration as well as a veterinary composition.

The disclosure also encompasses a vaccine comprising a recombinant strain according to this disclosure and a suitable carrier. This vaccine is especially useful for preventing or treating tuberculosis.

The recombinant cell according to this disclosure is also useful as a carrier for the expression and presentation of foreign antigens or molecules of interest that are of therapeutic or prophylactic interest.

Mycobacteria that may be employed to prepare the recombinant cell include but are not limited Mycobaceria such as M. microti, M. smegmatis, M. fortuitum, M. vaccae, M. hibemiae, M. terrae, M. triviale, M. triplex, genavense, M. kubicae, M. heidelbergense, M. cookii, M. haemophylum, M. botniense, M. conspicuum, M. doricum, M. farcinogenes, M. heckeshomense, M. monacense, M. montefiorense, M. murale, M. nebraskense, M. saskatchewanense, M. scrofulaceum, M. shimnodei, M. tusciae, M. xenopi, M. chelonae, M. boletii, M. peregrinum, M. porcinum, M. senegalense, M. houstonense, M. mucogenicum, M. mageritense, M. austroafricanum, M. diemhoferi, M. hodleri, M. frederiksbergense, M. aurum, M. chitae, M. fallax, M. confluentis, M. flavescens, M. madasgkariense, M. phlei, M. gadium, M. komossense, M. obuense, M. sphagni, M. agri, M. aichiense, M. alvei, M. arupense, M. brumae, M. canariasense, M. chubuense, M. duvalii, M. elephantis, M. gilvum, M. hassiacum, M. holsaticum, M. immunogenum, M. massiliense, M. moriokaense, M. psychrotolerans, M. pyrenivorans, M. vanbaalenii, M. pulveris, M. arosiense, M. aubagnense, M. chlorophenolicum, M. fluoranthenivorans, M. kumamotonense, M. novocastrense, M. parmense, M. phocaicum, M. poriferae, M. rhodesiae, M. seoulense, or M. tokaiense.

Methods of Inducing a Protective Immune Response Against M. tuberculosis

The disclosure encompasses methods for inducing an immune response against M. tuberculosis in a mammal. In some embodiments, the immune response is a protective immune response. The mammal may be a human Generally, the methods comprise administering an effective dose of a pharmaceutical composition of this disclosure to a mammal in an amount that induces an immune response, e.g., one that is protective against M. tuberculosis. The methods further comprise administering one or more exogenous agents so as to directly or indirectly activate and/or repress expression of toxic, e.g., bacteriocidal, gene products.

In methods comprising administering the pharmaceutical composition and the exogenous agent, the pharmaceutical composition and the exogenous agent may be administered simultaneously or at separate times. In embodiments in which the pharmaceutical composition and the exogenous agent are administered at separate times the order of administration may be (1) that the pharmaceutical composition is administered first and the exogenous agent is administered at a later point in time; or (2) that the exogenous agent is administered first and the pharmaceutical composition is administered at a later point in time, optionally along with the exogenous agent. In some embodiments the exogenous agent is administered second and is used to lyse the recombinant cell after a desirable result has been achieved. In some embodiments one or both of the pharmaceutical composition and the exogenous agent is administered at multiple timepoints.

In some embodiments the pharmaceutical composition is administered by an oral and/or subcutaneous and/or intradermal and/or inhalation and/or intravesicular mode. In some embodiments the pharmaceutical composition is intravenously administered.

Methods of Treating M. tuberculosis Infection

The disclosure encompasses methods for treating a M. tuberculosis infection in a subject. The subject may be any mammal. In some embodiments the subject is a human. Generally, the methods comprise administering an effective dose of a pharmaceutical composition to a subject infected with M. tuberculosis and inducing an immune response in the subject that ameliorates the M. tuberculosis infection. In one embodiment, after an immune response is induced, the exogenous agent is administered. In one embodiment, the exogenous agent is administered until an immune response is induced, after which the exogenous agent is no longer administered. In some embodiments, the M. tuberculosis infection is cured and the subject becomes free of M. tuberculosis. In some embodiments, the methods further comprise administering at least one anti-mycobacterial chemotherapeutic agent.

In methods comprising administering the pharmaceutical composition of this disclosure and a second agent (e.g., an anti-mycobacterial chemotherapeutic agent) to the subject, the pharmaceutical composition and the second agent may be administered simultaneously or at separate times. In embodiments in which the pharmaceutical composition and the second agent are administered at separate times the order of administration may be (1) that the pharmaceutical composition of this disclosure is administered first and the second agent is administered at a later point in time; or (2) that the second agent is administered first and the pharmaceutical composition of this disclosure is administered at a later point in time. In some embodiments the second agent is administered second. In some embodiments one or both of the pharmaceutical composition and the second agent is administered at multiple timepoints.

In some embodiments the pharmaceutical composition, the exogenous agent and/or the second agent (e.g., anti-mycobacterial chemotherapeutic agent) is administered by an oral and/or subcutaneous and/or intradermal and/or inhalation and/or intravesicular and/or intravascular mode. In some embodiments the pharmaceutical composition is intravenously administered and the exogenous agent and/or the second agent is orally administered.

In some embodiments the subject has an active tuberculosis infection. In some embodiments the subject has a latent tuberculosis infection.

### Cancer

The disclosure relates to a cancer treatment by immunotherapy with the recombinant strain, e.g., a recombinant BCG, as well as to a method for monitoring cancer treatment by immunotherapy with the recombinant strain.

It was recognized that long lasting direct contact with the live BCG resulted in tumor immunity (Zbar et al., 1971). Carcinoma of the bladder is the most common cancer of the urinary tract and the fourth most common malignant disease in the developed world (Jemal et al, 2011). Most tumors are diagnosed at a superficial stage and are surgically removed by transurethral resection (Babjuk. et al., 2011). Depending on the stage and grade of the non-muscle invasive tumors, adjuvant therapy is recommended as a strategy for both reducing recurrence and diminishing risk of progression. The initial treatment schedule was 120 mg lyophilized BCG Pasteur reconstituted in 50 mL saline and instilled via a catheter into the bladder. Patients were asked to retain the solution for at least 2 hours, and they additionally received 5 mg BCG intradermally. Treatments were given weekly over 6 weeks. Since then, BCG therapy has been the standard of care for high-risk urothelial carcinoma, namely carcinoma in situ, and high-grade Ta/T1 bladder lesions (Babjuk et al., 2011). It is also the most successful immunotherapy applied in the clinics, with response rates ranging 50-70% in patients with non-muscle invasive bladder cancer.

Modifications of the initial regimen have mainly focused on the elimination of the concomitant intradermal dose, introduction of maintenance BCG dosage schedule and introduction of other substrains of BCG. The regimen recommended by the European and American guidelines is as follows (Babjuk et al., 2011; Gontero et at, 2010): BCG therapy should be initiated 2 weeks after transurethral resection of the tumor. Any of the commercially available BCG strains (e.g. Connaught, Tice, RIVM) can be used for intravesical use. The routine procedure is to measure BCG dose in milligrams rather than in number of colony forming units (CFUs, i.e. live bacteria). Depending on the commercial preparation, dose range from 50 mg (Tice) to 120 mg (Pasteur) and are in the range of 10⁸-10⁹ CFUs. BCG dwell time in the bladder is 2 hours. The induction course of 6 weekly intravesical instillations is followed by maintenance therapy. The recommended maintenance regimen consists in 3 weekly instillations at 3 months, 6 months, and then every 6 months up to 3 years. The intravesical administration regimen recommended for BCG by the European and American guidelines comprises an induction course of 6 weekly intravesical instillations, followed by maintenance therapy. The recommended maintenance regimen consists in 3 weekly instillations at 3 months, 6 months, and then every 6 months up to 3 year. This administration regimen can be used for the composition in the treatment of bladder cancer.

In one embodiment, the composition for cancer immunotherapy may be delivered by parenteral or oral administration, e.g., at any time after cancer diagnosis. It is usually before tumor resection but can be concomitant with tumor resection. It may performed just after the diagnosis. It may be subcutaneous (s.c.), percutaneous, intradermal, local, intramuscular, oral, or intravenous. It may be one single administration. Local administration may be after tumor resection and at least seven days after the parenteral, e.g., intravenous, or oral administration. In one embodiment, it is at least three weeks after the parenteral or oral administration. The local administration at tumor site may depend on the type of cancer. For example, for bladder cancer it is intravesical. It usually comprises at least one series of at least three separate administrations, usually between three to six administrations, at an interval of one to three weeks. For the maintenance therapy, additional series of repeated administrations are generally performed using a similar administration regimen.

The methods of treatment, or the composition(s), may further comprise one or more additional agents like: (i) pro-inflammatory agents such as inflammatory cytokines (IL-2, IFNalpha, TNFalpha., GM-CSF), (ii) T-cell stimulatory molecules such as agonist antibodies directed against T-cell activating co-stimulatory molecules (CD28, CD40, OX40, GITR, CD137, CD27, HVEM) and blocking antibodies directed against T-cell negative co-stimulatory molecules (CTLA-4, PD-1, TIM-3, BTLA, VISTA, LAG-3), (iii) antibiotics, and/or (iv) chemotherapy drugs. Immunostimulatory molecules may be expressed from the vector.

Alternatively, the composition (s) comprising a non-pathogenic cell, e.g., a non-pathogenic or attenuated mycobacteria, may be used in combination (separate or sequential use) with such additional agents. For example, antibiotic(s) such as ofloxacin may be used in combination with the composition comprising live BCG or antigenically related non-pathogenic mycobacteria strain, to reduce side-effects in patients.

The composition(s) for use usually comprises a pharmaceutically acceptable carrier. The composition is further formulated in a form suitable for parenteral, oral, and/or local (e.g., intravesical, intravaginal, intratumor, or epicutaneous) administration into a subject, for example a mammal, and in particular a human.

Examples of cancer that can be treated using the treatment of the invention include with no limitation: bladder, melanoma, cervical, colon, prostate, ovarian and breast cancer.

The first composition comprising 10⁷ to 10⁹ CFUs of a recombinant bacteria may be injected intravascularly to intradermally (ID), e.g., to a PPD negative patient, shortly after a cancer diagnosis.

### Exemplary Systems for Trans Regulation

In one embodiment, a Lac repressor (Lad) and IPTG may be employed to control transcription of toxin genes.

In one embodiment, pristinamycin repressor (PipR) and pristinamycin may be employed to control expression of toxin genes.

In one embodiment, nitrilase repressor (NitR) and nitrile may be employed to control expression of toxin genes.

In one embodiment, cymene repressor (CymR) and cumate may be employed to control expression of toxin genes.

In one embodiment, arabinose repressor (AraC) and arabinose may be employed to control expression of toxin genes.

In one embodiment, the camphor repressor (CamR) and camphor may be employed to control expression of toxin genes.

In one embodiment, the Pseudomonas multidrug efflux repressor (PmeR) and parabens may be employed to control expression of toxin genes.

In one embodiment, the TtgR repressor and phloretin may be employed to control expression of toxin genes.

In one embodiment, xylose repressor (XylR) and xylose and may be employed to control expression of toxin genes.

In one embodiment, tetracycline repressor (TetR) and a tetracycline, e.g., short-acting tetracyclibes (half-life is 6-8 hours), e.g., tetracycline, chlortetracycline, oxytetracycline or olitetracycline, intermediate-acting (half-life is about 12 hours), e.g., demeclocycline or ethacycline, or tong-acting (half-life is 16 hours or more), e.g., doxycycline, minocycline, tigecycline or eravacycline, may be employed to control transcription.

Any repressor that is a sequence-specific DNA-binding protein (those that do not require a small-molecule regulator) may be employed in the OFF system, e.g., any of the repressors listed above as well as phage derived repressors like arc and mnt.

### Exemplary Cytocidal Gene Products

Exemplary cytocidal genes products which may be employed in the vectors and cells of this disclosure, cells including those from any organism including any microbe, e.g., virus or fungus, prokaryote or lower eukaryote, e.g., yeast, include but are not limited to kinases including avr (phosphorylates NAD), docP1 and/or docSM (both phosphorylate EF-Tu), EzeTC (phosphorylates UDP-N-acetylglucosamine), PezT (phosphorylates UDP-N-acetylglucosamine), and zetA (phosphorylates UDP-activated sugars), DNases, e.g. esaD, FicT, which amplifies GyrB, GhoT, which increases membrane permeability, lysins, e.g., phage lysins that may be specific for certain species of bacteria, or CRISPR-Cas9.

Lysins from phages that infect Mycobacterium include but are not limited to phages, e.g., from cluster A-X (see Table 1 in Hatfull, Microbiol. Spectr., 2018 6(5); 10.1128/microbiolsapec.GPP3-0026-2018, which is incorporated by reference herein. Exemplary mycobacterial phage lysins include but are not limited to LysA, LysB, e.g., D29 Lysin B, Hydrolase, e.g., glycoside hydrolase, peptidase, e.g., L-Ala-D-Glu peptidase, cysteine protease, muramidase, e.g., N-acetyl-β-D-muramidase, arabinogalactan esterase, lipase, or other toxins.

### Formulations and Routes of Delivery

### Uses

The modified (recombinant) cell can be used for administration to an individual for purposes of therapy or vaccination. Gene therapy can be conducted to enhance the level of expression of a particular protein either within or secreted by the cell. Vectors may be used to genetically alter cells either for gene marking, replacement of a missing or defective gene, or insertion of a therapeutic gene. Alternatively, a polynucleotide may be provided to the cell that decreases the level of expression. This may be used for the suppression of an undesirable phenotype, such as the product of a gene amplified or overexpressed during the course of a malignancy, or a gene introduced or overexpressed during the course of a microbial infection. Expression levels may be decreased by supplying a therapeutic or prophylactic polynucleotide comprising a sequence capable, for example, of forming a stable hybrid with either the target gene or RNA transcript (antisense therapy), capable of acting as a ribozyme to cleave the relevant mRNA or capable of acting as a decoy for a product of the target gene. Vaccination can be conducted to protect host organism from infection by pathogens including but not limited to Mycobacterium.

The introduction of the host cell to an animal may involve use of any number of delivery techniques (both surgical and non-surgical) which are available and well known in the art. Such delivery techniques, for example, include vascular catheterization, cannulization, injection, inhalation, endotracheal, subcutaneous, inunction, topical, oral, percutaneous, intra-arterial, intravenous, and/or intraperitoneal administrations. Vectors can also be introduced by way of bioprostheses, including, by way of illustration, vascular grafts (PTFE and dacron), heart valves, intravascular stents, intravascular paving as well as other non-vascular prostheses. General techniques regarding delivery, frequency, composition and dosage ranges of vector solutions are within the skill of the art.

In particular, for delivery of a vector of the invention to a tissue, any physical or biological method that will introduce the vector to a host animal can be employed. Vector means both a bare recombinant vector and vector DNA packaged into viral coat proteins, as is well known for administration. There are no known restrictions on the carriers or other components that can be coadministered with the vector (although compositions that degrade DNA should be avoided in the normal manner with vectors). Pharmaceutical compositions can be prepared as injectable formulations or as topical formulations to be delivered to the muscles by transdermal transport. Numerous formulations for both intramuscular injection and transdermal transport have been previously developed and can be used in the practice of the invention. The vectors can be used with any pharmaceutically acceptable carrier for ease of administration and handling.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of the host cell as a free acid (DNA contains acidic phosphate groups) or a pharmacologically acceptable salt can be prepared in water suitably mixed with a surfactant such as hydroxypropylcellulose. A dispersion of viral particles can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating the host cell in the required amount in the appropriate solvent with various of the other ingredients enumerated above, as required, followed by filtered sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the methods of preparation include but are not limited to vacuum drying and the freeze drying technique which yield a powder of the active ingredient plus any additional desired ingredient from the previously sterile-filtered solution thereof.

For purposes of topical administration, dilute sterile, aqueous solutions (usually in about 0.1% to 5% concentration), otherwise similar to the above parenteral solutions, are prepared in containers suitable for incorporation into a transdermal patch, and can include known carriers, such as pharmaceutical grade dimethylsulfoxide (DMSO).

Compositions may be used in vivo as well as ex vivo. In vivo gene therapy comprises administering the vectors directly to a subject. Pharmaceutical compositions can be supplied as liquid solutions or suspensions, as emulsions, or as solid forms suitable for dissolution or suspension in liquid prior to use. For administration into the respiratory tract, one mode of administration is by aerosol, using a composition that provides either a solid or liquid aerosol when used with an appropriate aerosolubilizer device. Another mode of administration into the respiratory tract is using a flexible fiberoptic bronchoscope to instill the vectors. Typically, the viral vectors are in a pharmaceutically suitable pyrogen-free buffer such as Ringer's balanced salt solution (pH 7.4). Although not required, pharmaceutical compositions may optionally be supplied in unit dosage form suitable for administration of a precise amount.

An effective amount of host cell is administered, depending on the objectives of treatment. An effective amount may be given in single or divided doses. Where a low percentage of transduction can cure a genetic deficiency, then the objective of treatment is generally to meet or exceed this level of transduction. In some instances, this level of transduction can be achieved by transduction of only about 1 to 5% of the target cells, but is more typically 20% of the cells of the desired tissue type, usually at least about 50%, at least about 80%, at least about 95%, or at least about 99% of the cells of the desired tissue type. The treatment can be repeated as often as every two or three weeks, although treatment once in 180 days may be sufficient.

The decision of whether to use in vivo or ex vivo therapy, and the selection of a particular composition, dose, and route of administration will depend on a number of different factors, including but not limited to features of the condition and the subject being treated. The assessment of such features and the design of an appropriate therapeutic or prophylactic regimen is ultimately the responsibility of the prescribing physician.

It is understood that variations may be applied to these methods by those of skill in this art without departing from the spirit of this invention.

### Dosages, Formulations and Routes of Administration

Administration of the recombinant host cells may be continuous or intermittent, depending, for example, upon the recipient's physiological condition, whether the purpose of the administration is therapeutic or prophylactic, and other factors known to skilled practitioners. The administration of the recombinant host cells may be essentially continuous over a preselected period of time or may be in a series of spaced doses. Both local and systemic administration is contemplated. When the recombinant host cells are employed for prophylactic or therapeutic purposes, recombinant host cells are amenable systemic administration.

One or more suitable unit dosage forms comprising the recombinant host cells, can be administered by a variety of routes including oral, or parenteral, including by rectal, transdermal, subcutaneous, intravenous, intramuscular, intraperitoneal, intrathoracic, intrapulmonary and intranasal routes. For example, intravenous administration may be desirable. For example, intradermal administration may be desirable. For administration to the lung, airway administration may be desirable. The formulations may, where appropriate, be conveniently presented in discrete unit dosage forms and may be prepared by any of the methods well known to pharmacy. Such methods may include the step of bringing into association the recombinant host cells with liquid carriers, solid matrices, semi-solid carriers, finely divided solid carriers or combinations thereof, and then, if necessary, introducing or shaping the product into the desired delivery system.

When the recombinant host cells are prepared for oral administration, they may be combined with a pharmaceutically acceptable carrier, diluent or excipient to form a pharmaceutical formulation, or unit dosage form. The total active ingredients in such formulations comprise from 0.1 to 99.9% by weight of the formulation. By “pharmaceutically acceptable” it is meant the carrier, diluent, excipient, and/or salt must be compatible with the other ingredients of the formulation, and not deleterious to the recipient thereof. The active ingredient for oral administration may be present as a powder or as granules; as a solution, a suspension or an emulsion; or in achievable base such as a synthetic resin for ingestion of the active ingredients from a chewing gum. The active ingredient may also be presented as a bolus, electuary or paste.

Pharmaceutical formulations containing the recombinant host cells can be prepared by procedures known in the art using well known and readily available ingredients. For example, the recombinant host cells can be formulated with common excipients, diluents, or carriers, and formed into tablets, capsules, suspensions, powders, and the like. Examples of excipients, diluents, and carriers that are suitable for such formulations include the following fillers and extenders such as starch, sugars, mannitol, and silicic derivatives; binding agents such as carboxymethyl cellulose, HPMC and other cellulose derivatives, alginates, gelatin, and polyvinyl-pyrrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium and magnesium stearate, and solid polyethyl glycols.

The recombinant host cells can also be formulated as elixirs or solutions for convenient oral administration or as solutions appropriate for parenteral administration, for instance by intramuscular, subcutaneous or intravenous routes.

The pharmaceutical formulations of the recombinant host cells can also take the form of an aqueous or anhydrous solution or dispersion, or alternatively the form of an emulsion or suspension.

Thus, the recombinant host cells may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol”, polyglycols and polyethylene glycols, C₁-C₄ alkyl esters of short-chain acids, e.g., ethyl or isopropyl lactate, fatty acid triglycerides such as the products marketed under the name “Miglyol”, isopropyl myristate, animal, mineral and vegetable oils and polysiloxanes.

For administration to the upper (nasal) or lower respiratory tract by inhalation, the recombinant host cells are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the recombinant host cells may be administered via nose drops, a liquid spray, such as via a plastic bottle atomizer or metered-dose inhaler. Typical of atomizers are the Mistometer (Wintrop) and the Medihaler (Riker).

The local delivery of the recombinant host cells can also be by a variety of techniques. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

Drops, such as eye drops or nose drops, may be formulated with an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The formulations and compositions described herein may also contain other ingredients such as antimicrobial agents, or preservatives. Furthermore, the active ingredients may also be used in combination with other agents, for example, bronchodilators.

The recombinant host cells may be administered to a mammal alone or in combination with pharmaceutically acceptable carriers. As noted above, the relative proportions of active ingredient and carrier are determined by the solubility and chemical nature of the compound, chosen route of administration and standard pharmaceutical practice.

The dosage of the recombinant host cells will vary with the form of administration, the particular compound chosen and the physiological characteristics of the particular patient under treatment.

### Exemplary Embodiments

In one embodiment, the disclosure provides a vector system. In one embodiment, a vector comprises at least two expression cassettes, wherein a first expression cassette comprises a first transcriptional regulatory region that includes a promoter operably linked to a first open reading frame encoding a first bacteriocidal gene product, wherein transcription from the first transcriptional regulatory region is controlled in trans by a first transcriptional regulatory protein, wherein a second expression cassette comprises a second transcriptional regulatory region that includes a promoter operably linked to a second open reading frame encoding a second bacteriocidal gene product, wherein transcription from the second transcriptional regulatory region is controlled in trans by a second transcriptional regulatory protein, wherein the first and second open reading frames encode different bacteriocidal gene products, wherein the activity of at least the first transcriptional regulatory protein is controlled by a first exogenous agent, and wherein optionally the vector further comprises a third expression cassette comprising a third transcriptional regulatory region that includes a promoter operably linked to a third open reading frame encoding the second transcriptional regulatory protein, wherein transcription from the third transcriptional regulatory region is controlled in trans by a third transcriptional regulatory protein, wherein the activity of the third transcriptional regulatory protein is controlled by the exogenous agent. In one embodiment, the vector further comprises nucleic acid encoding the first transcriptional regulatory protein. In one embodiment, the vector further comprises nucleic acid encoding the first transcriptional regulatory protein and nucleic acid encoding the third transcriptional regulatory protein. In one embodiment, the first and the second transcriptional regulatory regions are controlled by the same transcriptional regulatory protein. In one embodiment, the activity of the second transcriptional regulatory region is controlled by the first exogenous agent. In one embodiment, the activity of the second transcriptional regulatory region is controlled by a different exogenous agent than the first transcriptional regulatory region. In one embodiment, the exogenous agent comprises a tetracycline, a streptogramin or a macrolide, e.g., erythromycin, clarithromycin, or roxithromycin. In one embodiment, the presence of the exogenous agent induces expression of the first and the second bacteriocidal gene products from the vector. In one embodiment, the exogenous agent binds the first transcriptional regulatory protein. In one embodiment, the first and the second transcriptional regulatory regions are controlled by different transcriptional regulatory proteins. In one embodiment, the vector comprises the third expression cassette and in one embodiment, the presence of the exogenous agent represses expression of the first and the second bacteriocidal gene products from the vector. In one embodiment, the first transcriptional regulatory protein and the second transcriptional regulatory protein comprise TetR, EryR, PipR CamR, PmeR, TtgR, XylR, Mnt, Arc or other proteins that can serve as transcriptional regulators in the host carrying the kill switch system. In one embodiment, the first transcriptional regulatory protein comprises revTetR and wherein the second transcriptional regulatory protein comprises a DNA binding protein that represses transcription, e.g., PipR. In one embodiment, the first transcriptional regulatory protein comprises revTetR and wherein the third transcriptional regulatory protein comprises TetR. In one embodiment, the first open reading frame or the second open reading frame encode a lysin or a nuclease. In one embodiment, the first open reading frame and the second open reading frame encode different lysins. In one embodiment, the first open reading frame encodes a lysin and the second open reading frame encodes a nuclease. In one embodiment, the lysin is a murein hydrolase. In one embodiment, the first open reading frame and the second open reading frame encode different nucleases. In one embodiment, the expression cassettes are on a plasmid. In one embodiment, one of the expression cassettes is on one plasmid and the other expression cassette is on another plasmid or is part of a host cell genome. In one embodiment, a host cell expressing the first or third transcriptional regulatory protein is transfected with a vector having the first and second expression cassettes or the first and third expression cassettes, respectively. In one embodiment, the host cell constitutively expresses the first or third transcriptional regulatory protein. In one embodiment, the host cell or the vector system includes nucleic acid encoding an immunostimulatory gene product (e.g., antigen 85A/B, ESAT6, GroEL or other antigenic, immunostimulatory Mtb proteins; or immunostimulatory human proteins such as interleukins or interferons). In one embodiment, the vector further comprises one or more genes of interest.). In one embodiment, the vector does not include recombination sites. In one embodiment, the first or the second bacteriocidal gene product in a recombinant cell having the vector(s) is a heterologous bacteriocidal gene product, e.g., the bacteriocical gene product is not naturally found in the species of cell from which the recombinant cell was prepared or derived. In one embodiment, the first or the second bacteriocidal gene product in a recombinant bacterial cell having the vector(s) is a phage encoded gene product, e.g., a phage lysin. In one embodiment, the first or the second bacteriocidal gene product in a recombinant cell having the vector(s) is a non-secreted gene product. In one embodiment, the first and the second bacteriocidal gene products in a recombinant cell are each a heterologous bacteriocidal gene product. In one embodiment, the first and the second bacteriocidal gene products in a recombinant cell having the vector(s) are each a phage encoded gene product. In one embodiment, the first and the second bacteriocidal gene products in a recombinant cell having the vector(s) are each a non-secreted gene product. In one embodiment, the first or the second bacterocidal gene product is a mycobacterial phage encoded gene product. In one embodiment, the first and the second bacterocidal gene products are each a mycobacterial phage encoded gene product.

In one embodiment, the disclosure provides an isolated recombinant cell having the disclosed vector. In one embodiment, the recombinant cell is an attenuated microbial cell, e.g., a microbial cell that has reduced pathogenicity after infecting a host organism. In one embodiment, the recombinant cell is a bacterial cell. In one embodiment, the recombinant cell is a fungal cell. In one embodiment, the recombinant cell is a yeast cell. In one embodiment, the recombinant cell is a plant cell. In one embodiment, the recombinant cell is a mammalian cell. In one embodiment, the vector or the recombinant cell do not comprise sequences encoding an antibiotic resistance gene product. In one embodiment, the recombinant cell is a Mycobacterium. In one embodiment, the cell is an attenuated Mycobacterium. In one embodiment, the cell is M. bovis. In one embodiment, the cell is BCG cell. In one embodiment, prior to introduction of the vector having at least two expression cassettes, the cell includes sequences encoding the first transcriptional regulatory protein. In one embodiment, prior to introduction of the vector, the cell expresses the first transcriptional regulatory protein, e.g., constitutively expresses the regulatory protein. In one embodiment, the expression of the first transcriptional regulatory protein in the cell is inducible.

In one embodiment, prior to introduction of the vector having three expression cassettes, the cell includes sequences encoding the first and the third transcriptional regulatory proteins. In one embodiment, prior to introduction of the vector, the cell constitutively expresses the first or third transcriptional regulatory protein. In one embodiment, the expression of the first or the third transcriptional regulatory protein in the cell is inducible. Further provided is a composition comprising the disclosed vector or the disclosed recombinant cell. In one embodiment, the composition has an amount of the disclosed recombinant cell effective to induce an immune response to the cell.

In one embodiment, a method to control expression of linked genes in a recombinant cell is provided. In one embodiment, the disclosed recombinant cell is contacted with at least one exogenous agent in an amount that alters expression from the first transcriptional regulatory region and the second transcriptional regulatory region relative to expression in the absence of the exogenous agent. In one embodiment, the disclosed recombinant cell is contacted with at least one exogenous agent in an amount that alters expression from the first transcriptional regulatory region and the third transcriptional regulatory region relative to expression in the absence of the exogenous agent. In one embodiment, the amount of the exogenous agent increases expression from the first and the second transcriptional regulatory region. In one embodiment, the amount of the exogenous agent represses expression from the first transcriptional regulatory region and enhances expression from the third transcriptional regulatory region. In one embodiment, the exogenous agent is a tetracycline or a macrolide. In one embodiment, the cell is a BCG strain.

Also provided is a method to induce an immune response to Mycobacterium in a mammal In one embodiment, a mammal is administered an amount of the disclosed recombinant cell effective to induce an immune response to Mycobacterium in the mammal and is administered an amount of the exogenous agent effective to control viability of the recombinant cell. In one embodiment, the mammal is infected with Mycobacterium prior to administration of the recombinant cell and/or the exogenous agent. In one embodiment, the mammal is not infected with Mycobacterium prior to administration of the recombinant cell and/or the exogenous agent. In one embodiment, the administration of the exogenous agent increases expression of the first and the second bacteriocidal gene products encoded by the vector in the recombinant cell. In one embodiment, the exogenous agent is administered in an amount effective to lyse the cell. In one embodiment, the administration of the exogenous agent represses expression of the first and the second bacteriocidal gene products encoded by the vector in the recombinant cell. In one embodiment, the exogenous agent is administered in an amount effective maintain the viability of the recombinant cell in the mammal In one embodiment, the amount of administered exogenous agent is decreased or omitted so that the first and the second bacteriocidal gene products are expressed. In one embodiment, the recombinant cell is intravenously administered. In one embodiment, the recombinant cell is intradermally administered. In one embodiment, the recombinant cell is delivered to the lungs, e.g., as an aerosol.

In one embodiment, a method to inhibit or treat bladder cancer in a mammal is provided. In one embodiment, a mammal in need thereof is administered an amount of the recombinant cell effective to inhibit or treat bladder cancer in the mammal and is administered an amount of the exogenous agent effective to control viability of the cell. In one embodiment, the recombinant cell is intravenously administered. In one embodiment, the recombinant cell is intradermally administered. In one embodiment, the mammal is a human In one embodiment, the mammal has been subjected to transurethral resection of a bladder tumor. In one embodiment, the recombinant cell is administered once a week over 6 to 12 weeks.

The invention will be described by the following non-limiting examples.

### EXAMPLE 1

To deliver attenuated strains of BCG that may be a safer alternative vaccine for intravenous or other forms of delivery, mycobacterial strains that, in one embodiment, grow relatively normally in the presence of specific small molecules but die fairly rapidly when those molecules are withdrawn were prepared. This allows for safer strains, incapable of causing disease, but also allows the total bacterial load in vivo to be set by titrating the appropriate small molecules.

In one embodiment, the attenuated BCG strain is “addicted” to a small molecule. These strains can only grow in the presence of an exogenously added small molecule. Upon withdrawal of that molecule, bacteria die because of the production of a toxin (a bacteriocidal gene product).

Toxic kill switch. For this strategy transcriptional regulation of toxic molecules is employed. The general strategy is illustrated in FIG. 1. In this embodiment, a tetracycline-regulated promoter is employed. The promoter is inactive (off) in the presence of a tetracycline analogue (e.g., anhydrotetracycline (ATC) for in vitro experiments, doxycycline in vivo) but becomes active when the tetracycline is withdrawn. In this approach, the promoter drives the expression of two different toxin (bacteriocidal) genes. Exemplary toxin genes include various bacteriophage-derived lysins, a DNase or an RNase. Expression of any of these genes results in cell death, though the kinetics of death and the rate at which mutations result in survival (the escape frequency) may vary depending on the toxin gene and the specific characteristics of the promoter.

This approach employs DNA encoding a “dual-toxin on/off switch” that makes other genes, e.g., in one embodiment, the genes are on the same plasmid, constitutively active and constitutively turned off by administration of an agent (“on) or active only in the presence of said agent (”off”). In one embodiment, the “dual-toxin on/off switch” employs sequences from two different lysin operons. In one embodiment, the lysin operons are derived from phages. In one embodiment, a plasmid includes other genes as well as the “dual-toxin on/off switch.” The plasmid allows for a method to control gene expression using the “dual-toxin on/off switch” and a separate agent (exogenously administered) that activates the switch. In one embodiment, the exogenous agent is a tetracycline, including but not limited to doxycycline or anhydrotetracycline. In one embodiment, the host cell having the plasmid with the “dual-toxin on/off switch” is a BCG strain. In one embodiment, the “dual-toxin on/off switch” is first turned “on” and then is turned off by the exogenous agent (e.g.,“TetON”). In one embodiment, the “dual-toxin on/off switch” is turned off and then is turned on by the exogenous agent (e.g., it is addicted to the agent) (e.g., “TetOFF”). The use of such a switch, e.g., in a host cell that is administered to a host organism, allows for a method to vaccinate against TB comprising administering the host cell and subsequently administering an agent that turns off expression of BCG (lyses BCG). In one embodiment, the switch is used in a method to treat bladder cancer comprising administering the host cell and subsequently administering an exogenous agent that turns off BCG (lyses BCG) and so the BCG cannot cause infection. In one embodiment, the use of a switch allows for a method to vaccinate against TB comprising administering the host cell along with an exogenous agent that keeps the host cell active (viable) (which would be safer than current live BCG vaccines); in this case the BCG will die after the agent is all metabolized. In one embodiment, the use of a switch provides for a method to treat bladder cancer comprising administering the host cell along with an exogenous agent that keeps the host cell active viable) until all of the exogenous agent is metabolized.

Engineered BCG should be similarly or even less virulent than wild type BCG when grown in the presence of addictive molecules. In their absence (“OFF”) the engineered strains rapidly die. However, strains can accumulate mutations that could allow them to grow in the absence of the addictive molecule. Under those circumstances those cells are no more virulent than wild type BCG. The rate of these “escape” mutations is likely in the range of 10⁶-10⁸.

### Methods

For genomic engineering, ORBIT, a recombineering method that takes advantage of two phage enzymes, is used to either disrupt a chromosomal gene or insert a coding domain. This requires three steps—transformation of a recombinase plasmid, introduction of the recombinatorial substrate, and curing of the recombinase plasmid. The introduction of new genes may be conducted in a single step to introduce kill switches. The removal of antibiotic markers may be accomplished in two or more steps, e.g., via introduction of a recombinase plasmid followed by curing of the plasmid.

Characterization of escape rates is performed on final strains by either measuring frequency or rate determined by fluctuation analysis using the Luria procedure. Furthermore, vaccine efficacy of M. bovis BCG in non-human primates (NHP) is dependent on the route of administration. Intravenous application of a relatively high dose of M. bovis BCG is superior to low-dose intradermal injection that mimics how the vaccine is currently administered in humans. The conditionally replicating BCG strains disclosed herein are safer and so are particularly useful in TB vaccination and bladder cancer therapy.

M. bovis BCG is a live vaccine while the disclosed BCG-strains conditionally grow with (e.g., TetOFF) or without (e.g., TetON) a tetracycline. To test the vaccine strains, a conditionally replicating Mtb strain that is suitable as a challenge strain in a controlled human infection model for TB may be employed. Production of volatiles and fluorescent proteins allows for quantification and detection of the challenge strain in deep tissue and skin, respectively. If needed, other attenuation mechanisms may be employed in the dual toxin on/off strains, e.g., mechanism that provide for 10⁻¹⁴ escapes/generation although 10-8 escapes/generation may be safe enough to use.

### EXAMPLE 2

### TetON Double Lysin Strain

An inducible kill switch was added to BCG, where an ATC-inducible regulation system controls expression of a lysin gene, so that upon removal of ATC the lysin is expressed and the cell is killed by lysis. The final strain is: Strain 2071: pGMCK3-TSC10M-TsynC-pipR-SDn-P1-TsynE-PptR-L5L, pGMCgZni-TSC38S38-TrrnBd2-P749pld-10C32C8C-D29L. The strain uses the same type of TetR and the same TetR-regulated promoter in both plasmids. The differences are (i) antibiotic marker, (ii) integration site into the chromosome, and (iii) lysin cassette (one derived from the phage L5, the other from the phage D29).

The inducible lysing strain is likely safer than the WT strain since the viability of the bacteria is controlled, e.g., it is eliminated faster due to the double toxin system. And bacterial lysis could lead to adequate exposure of bacterial components and antigens for better immunogenicity. Since it is safer, it could be used in other immunization methods such as iv.

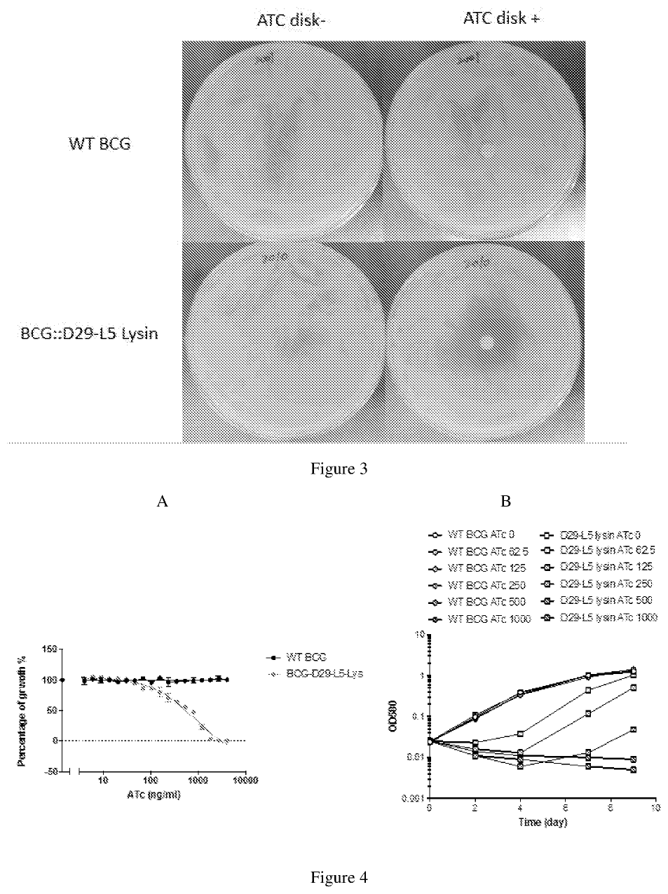

FIG. 3 shows the use of the TetON system to induce lysin expression. Without ATC, the tet repressor (TetR) would bind the tet operator (tetO) and inhibit gene expression whereas, in the presence of ATC, ATC would bind TetR and the target gene would be expressed by the promoter. In this example, D29 and L5 lysins were respectively expressed on L5 site and giles site.

FIG. 4 provides data from the ATC disk assay which indicates that growth of a double lysin expressing TetON strain was effectively inhibited in the presence of ATC and the ATC had no effect on WT BCG strain.

After constructing the strains, the sensitivity of the strains to ATC was tested in 7H9 broth medium. MIC curve and growth curves were set up with WT and double lysin strain with different ATC concentrations. FIG. 5 shows that the double lysin strain was sensitive to ATC in a dose dependent pattern whereas the WT strain was not influenced by presence of ATC. 1000 ng/ml ATC efficiently inhibited the bacterial growth without causing any growth defect in the WT strain so this concentration was employed in some experiments.

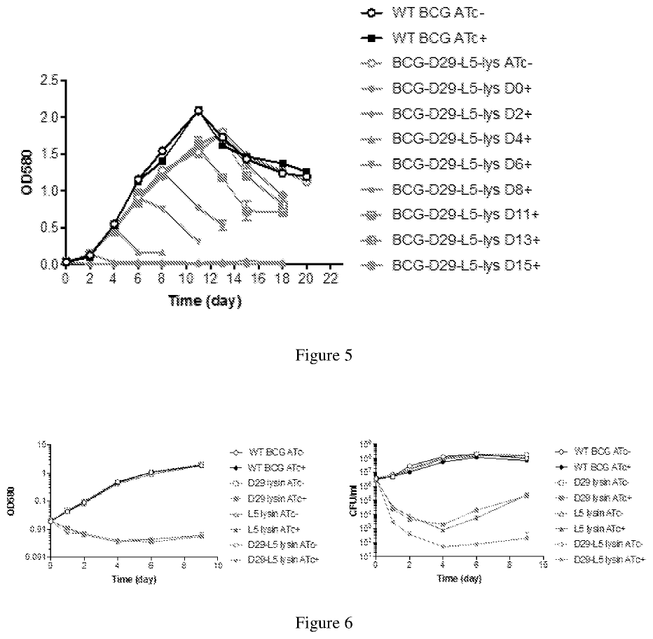

ATC was added at different times and growth and culture OD measured. Both growth and culture OD dropped quickly once ATC was added (FIG. 6). This means that lysin expression was well regulated by ATC and the expression of two lysins could efficiently lyse the bacteria cell. The CFUs dropped quickly upon exposing the infected cells to ATC. The CFUs dropped 4-5 logs in 4 days.

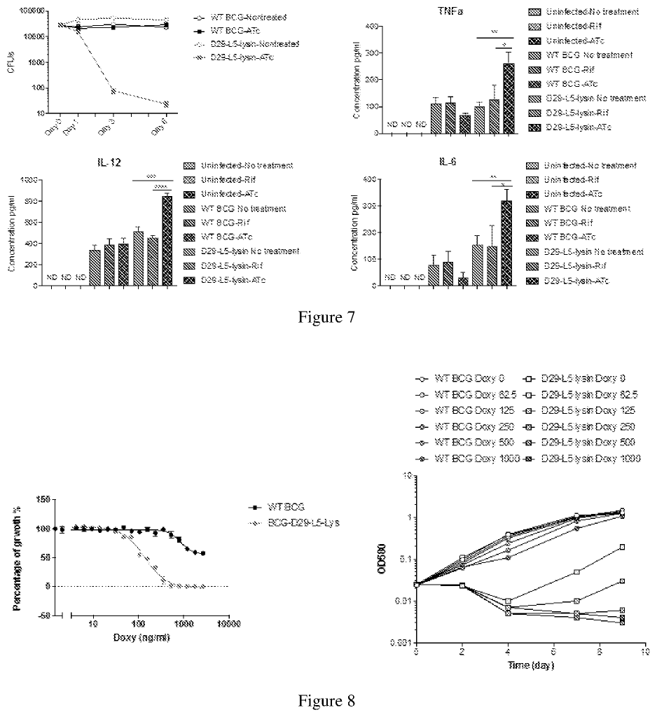

The killing effect of lysins was measured in infected macrophage. Bone marrow derived macrophages were isolated and infected with WT and double lysin BCG strains. After infection, macrophages were treated with ATC, rifampin or non-treated. Double lysin strain CFUs decreased following ATC treatment (FIG. 7). ATC treatment in the double lysin strain induced more proinflammatory cytokine production including TNFa, IL-12 and IL-6 (FIG. 7).

Doxycycline sensitivity of WT and double lysin strains was measured with a MIC curve and growth curve assay (FIG. 8). BCG is more sensitive to doxycycline than ATC. Growth of both WT BCG and double lysin strains were inhibited by high concentrations doxycycline. However, the double lysin strain is more sensitive to doxycycline than WT BCG.

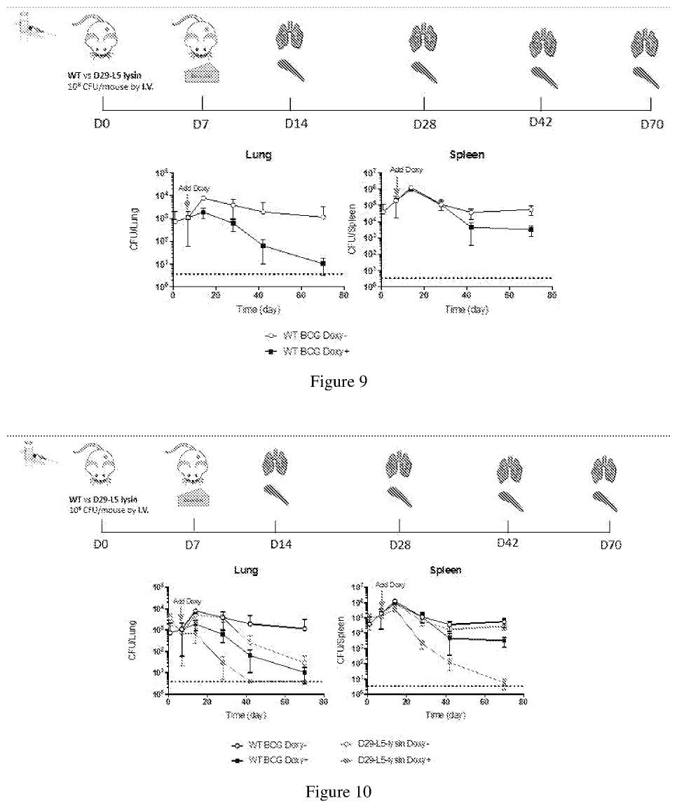

A mouse infection model was used to test the lysis of the strains in vivo (FIGS. 9-10). WT mice were infected with WT and double lysin BCG strains by i.v. administration. Mice were fed a diet having doxycycline starting at day 7 post-infection. Then bacterial burden in lung and spleen was evaluated. From the in vitro MIC and growth curve data, high concentration of doxycycline suppressed WT BCG. Doxycycline also reduces WT BCG CFUs in vivo in infected mice. Since doxycycline concentrations in the lung are higher than spleen, the CFUs in lung decreased faster than spleen. CFUs decreased faster in the double lysin strain than the WT strain in both lung and spleen. So doxycycline treatment led to double lysin strain bacterial death in the mouse infection model.

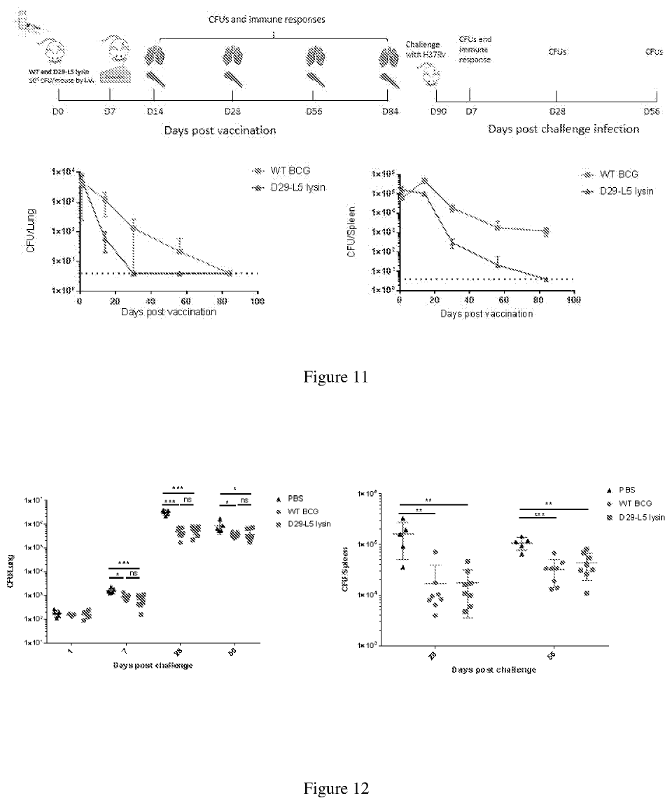

To determine if inducing bacteria lysis could result in protection efficacy compared to WT BCG, mice were vaccinated with WT and double lysin BCG by i.v., then lysis was induced by feeding the mice doxycycline. During the lysis process, samples were harvested to monitor BCG CFUs and immune responses of the mice. After 90 days, the mice were infected with H37Rv and MTB and bacteria burden at D7, D28 and D56 determined (FIGS. 11-12). The BCG bacteria load was as expected, and the double lysin strain was killed faster in both lung and spleen. WT BCG was cleared in the lung, but around 1000 CFU still existed right before rechallenge.

Although the double lysin strain induced the same protection as WT strain, considering that the double lysin strain was cleared and there were still 1000 CFU of WT BCG, the double lysin strain may be a safer strain, e.g., when used i.v.

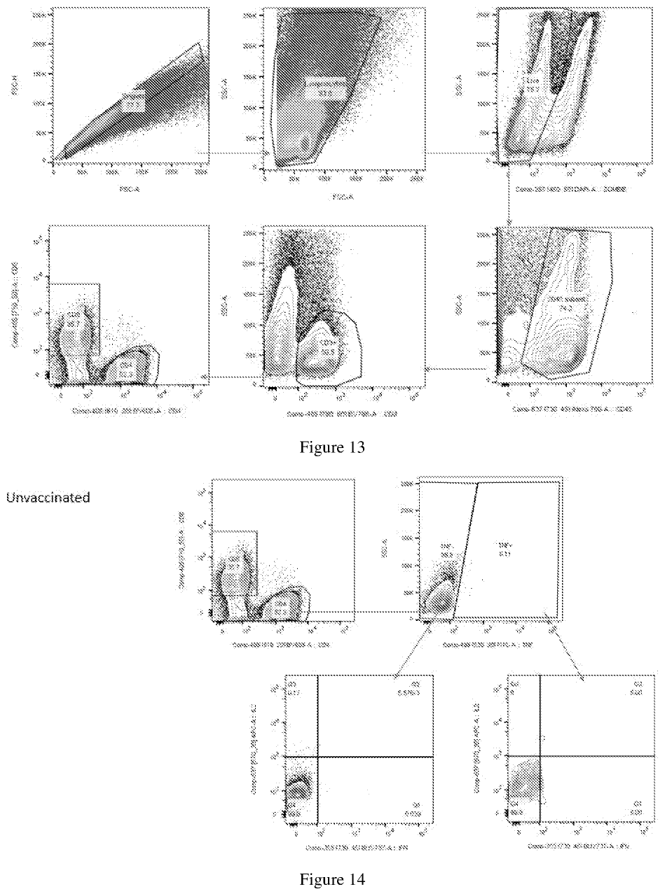

FIG. 13 shows a general gating strategy to identify helper and cytotoxic T cells. Singlets and lymphocytes were first gated out, then live cells were isolated with zombie staining negative population. Then CD45 and CD3 positive cells were used to isolate T cells, and CD4 and CD8 positive cells were isolated from CD3 positive cells to provide helper and cytotoxic T cells. From CD4 T cells, a TNFa positive and negative population is gated out, then IFN gamma and IL-2 positive and negative populations from these two populations were gated out.

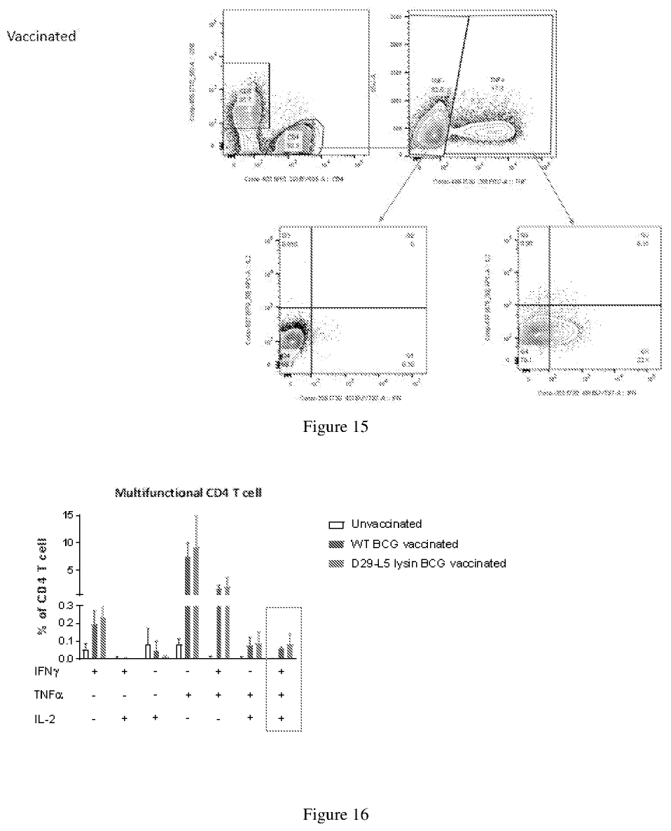

In the unvaccinated example (FIG. 14), rarely are there any cytokine positive populations. In the vaccinated example (FIG. 15) there is a big population of TNFa positive cells, and even a double positive and a triple positive population. Thus, i.v. vaccination of both WT and double lysin strains induced more double and triple cytokine production population.

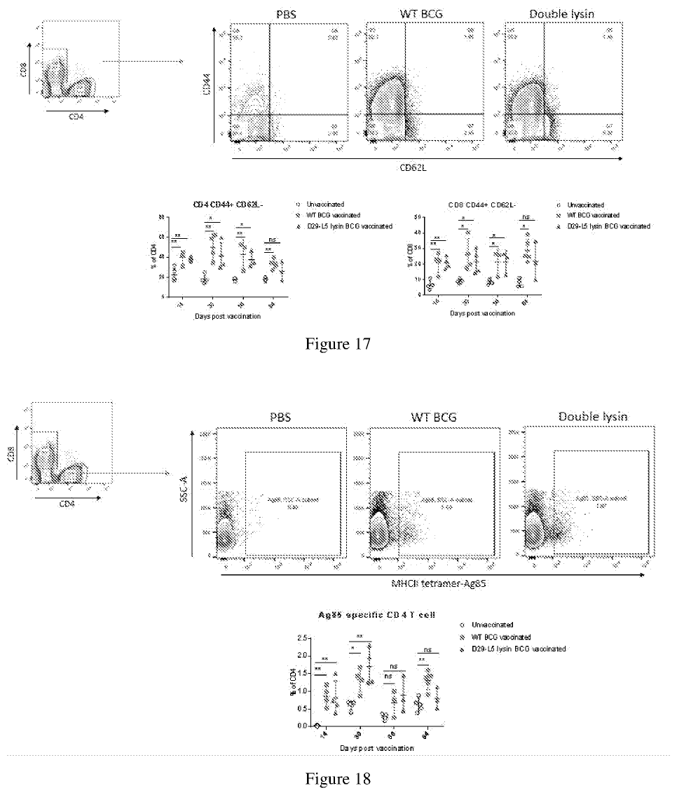

Both CD4 and CD8 effector memory T cells had high levels of expression of CD44 and a low level of expression of CD62L which constantly increased in the vaccinated group during vaccination (FIGS. 16-17).

By utilizing MHC tetramer of Ag85, Ag85 specific CD4 T cells could be detected. There were more Ag85 specific CD4 T cells detected in the vaccinated group (FIG. 18).

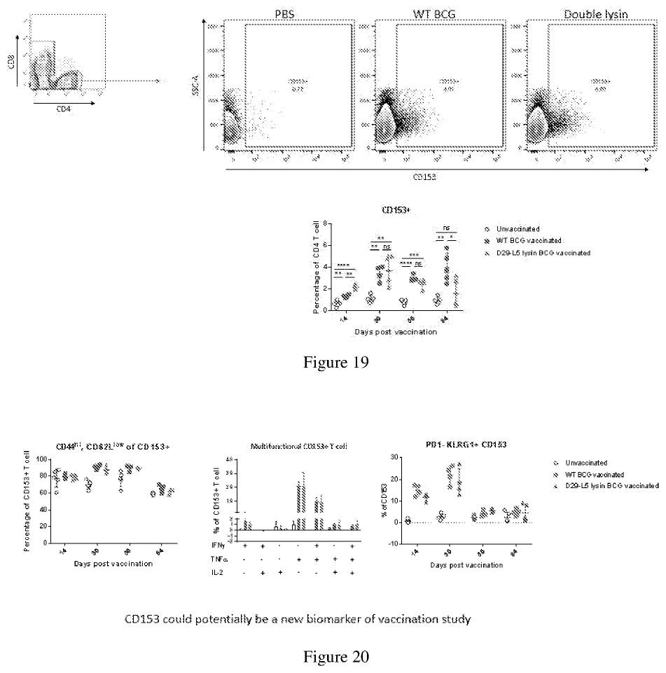

CD153+CD4+ T cells may be required for mice to control pulmonary MTB infection as the mice succumb quickly to MTB infection without this molecule. CD153+CD4+ T cells are located in parenchyma. FIG. 19 shows data for this population during vaccination. There is an increase in WT and double lysin BCG strains compared to unvaccinated mice. And this increase is constant and long lived even after the bacteria load is decreased.

The characteristics of CD153 positive CD4 T cells were analyzed (FIG. 20). Around 80% of the population was effector memory CD4 T cells with low levels of CD62L expression and high levels of CD44 expression even in the unvaccinated group. A high proportion of these cells are double and triple cytokine production CD4 T cells upon PPD stimulation. A small proportion of these cells are KLRG1+ even when the bacterial load is high; it decreased to just 1% after 56 days. This indicates that the population is located in lung parenchyma. So CD153 could be a new biomarker of vaccination as well as a marker of lung resident memory CD4 T cells

### EXAMPLE 3

### TetOFF Double Lysin Strain

An inducible lysin strain would be safer than the WT strain since the viability of the bacteria could be controlled. And bacterial lysis could lead to adequate exposure of bacterial components and antigens, thereby resulting in better immunogenicity. Since the double lysin strain is safer, it could be used in other immunization methods such as iv. However, considering that people may need to take a tetracycline such as doxycycline for months to induce lysis and clear bacteria, a TetOFF system was designed.

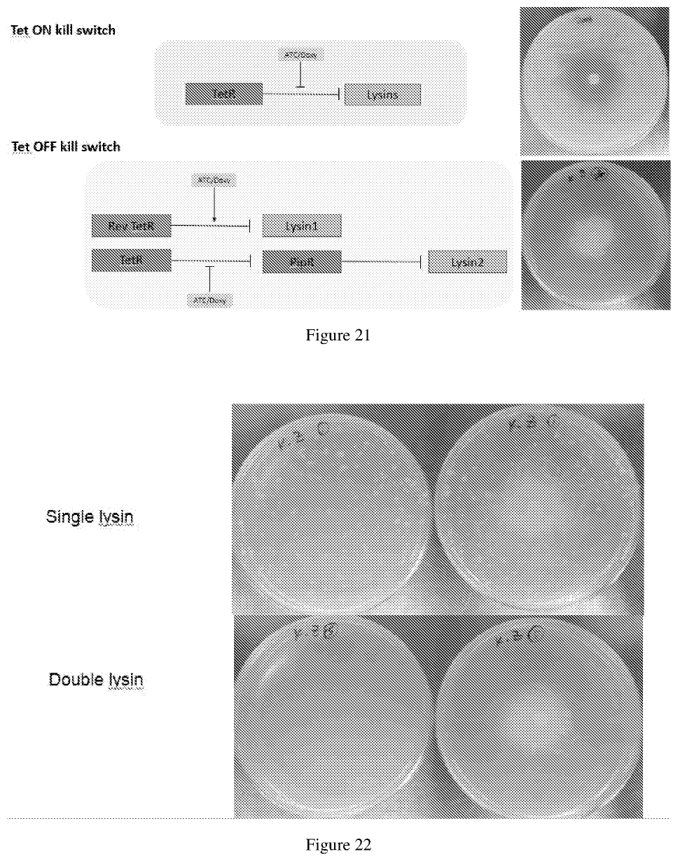

FIG. 21 shows exemplary vectors for TetOFF. The TetOFF kill switch system induces lysis without administering a tetracycline, e.g., doxycycline. Reverse tetR was used to suppress lysinl in the presence of ATC/Doxycycline. TetR was used to control pipR, a repressor controlling lysin2. In the presence of ATC, Reverse tetR suppresses lysin1 expression. And TetR induces pipR expression which suppresses lysin2 expression. In the absence of ATC, lysin and lysin2 are induced to be expressed at the same time. This may effectively decrease escape mutants since mutations overcoming reverse tetR and TetR or mutations overcoming lysin activity would be needed.

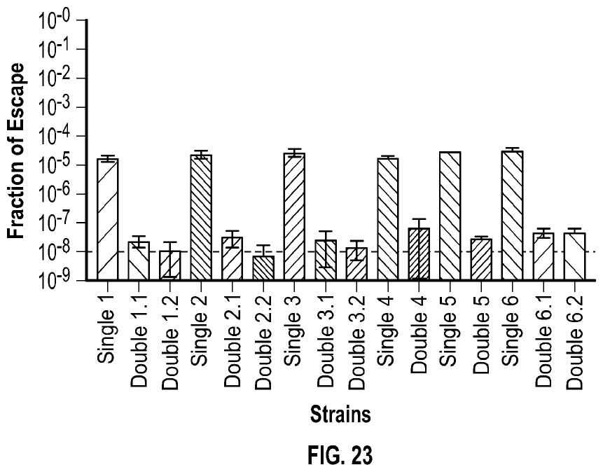

FIG. 22 shows data for an example of TetOFF kill switch with ATC disk assay (single and double lysin). Bacteria can grow around the ATC disk and could not grow without ATC. FIG. 23 shows that the double lysin system decreased escape mutants compared with the single lysin strain.

In order to identify the best strain, strains combining different promoters driving lysin expression were constructed and their fraction of escape mutants determined. Thus, the double lysin strain has consistently less suppressors.

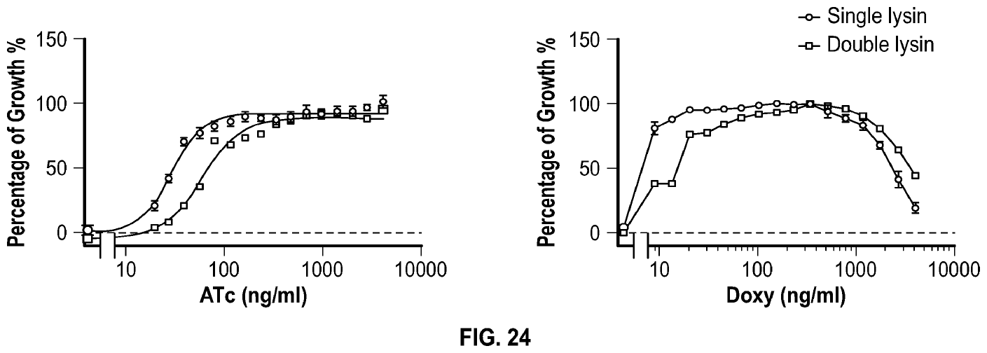

ATC and doxycycline sensitivity were tested in a MIC like assay. With increasing ATC and doxycycline concentrations (FIG. 24), bacterial growth was stimulated and bacteria could not grow without ATC or doxycycline. Since BCG is sensitive to doxycycline, growth was inhibited at high concentrations.

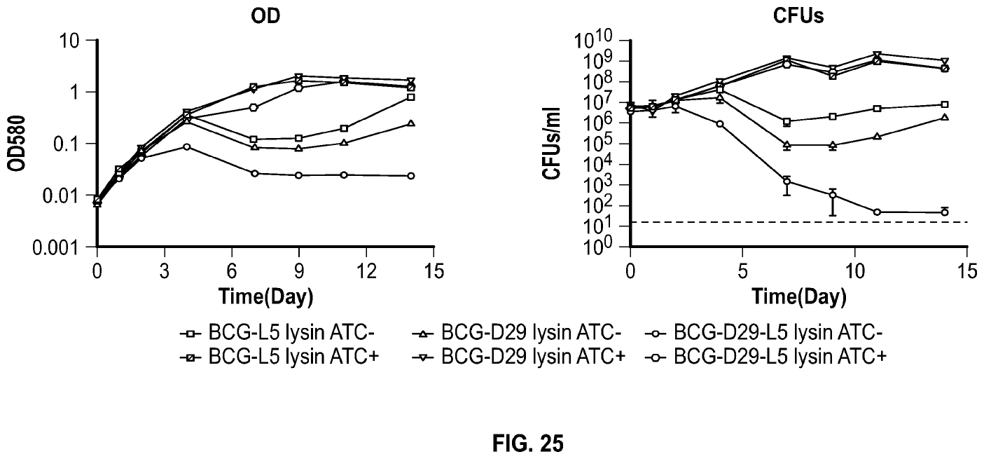

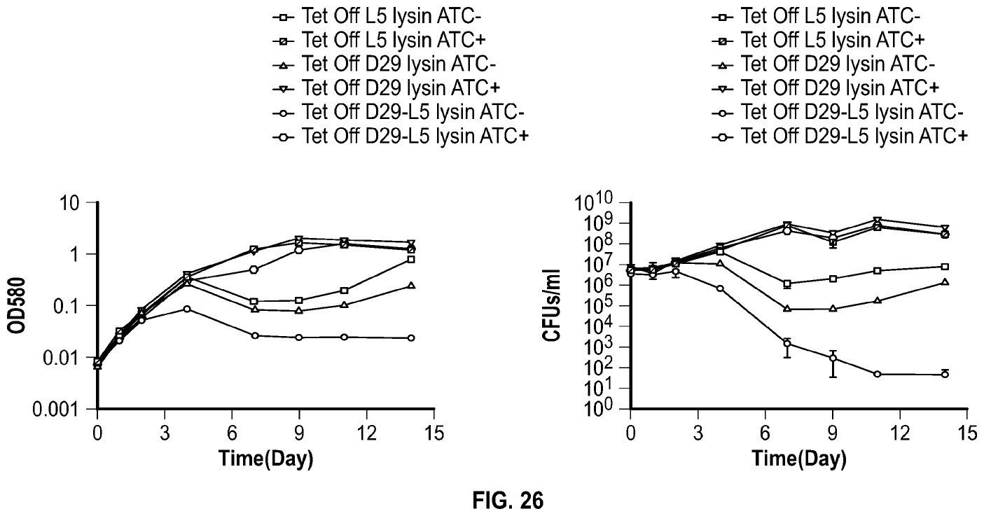

Killing curve experiments were used to test the killing kinetics of a TetOFF dual lysin strain (FIG. 25). After two days incubating without ATC, bacterial CFUs started to decrease and decreased by 6 logs in around 10 days. The double lysin strain was killed faster than the single lysin strains.

A fluctuation assay was used to evaluate the resistance frequency to toxin stress. 20 independent cultures were started from a very low inoculum and allowed to grow around 14 days to OD 0.8-1. Escape mutant numbers were then determined for each culture. The mutation rate was calculated through a mathematic model. The mutation rate per cell per division is around 4-7×10⁻⁹;

### Methods and Results for TetOFF Double Lysin Strain

Strain number and name: 2071-BCG TetOFF dual lysin strain; strain background is BCG Pasteur from ATCC.

### Plasmids for Lysin Expression:

pGMCK3-TSC10M-TsynC-pipR-SDn-P1-TsynE-PptR-L5L,
pGMCgZni-TSC38S38-TrrnBd2-P749pld-10C32C8C-D29L

### Growth Condition:

7H9 medium containing the following antibiotics:
Kanamycin, 25 ug/ml
Zeocin, 25 ug/ml
ATC, 1000 ng/ml

### Fraction of Escape:

Fraction of escape is approximately 10⁻⁸.
Fluctuation analysis indicated that the resistance rate per cell per generation is 2×10⁻⁹-4×10⁻⁹.

### Induction of Bacterial Lysis in Liquid 7H9 Medium

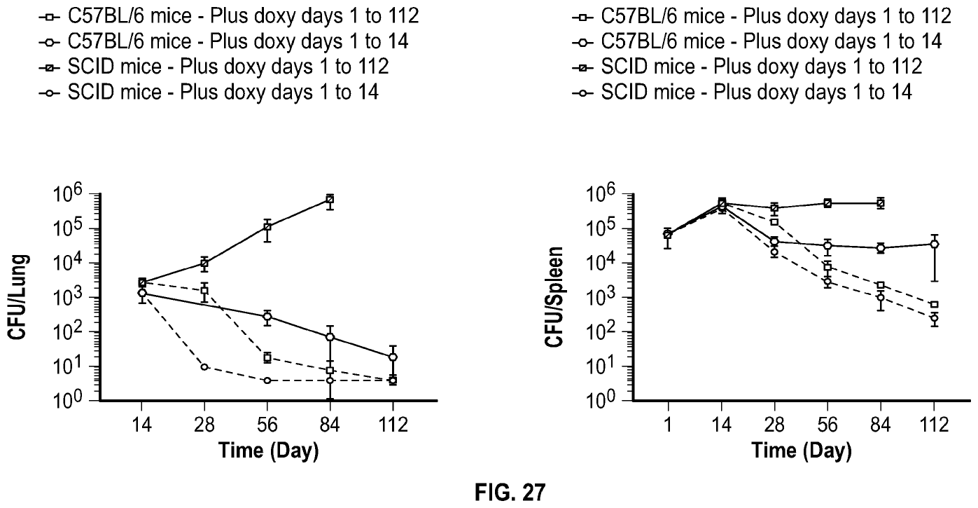

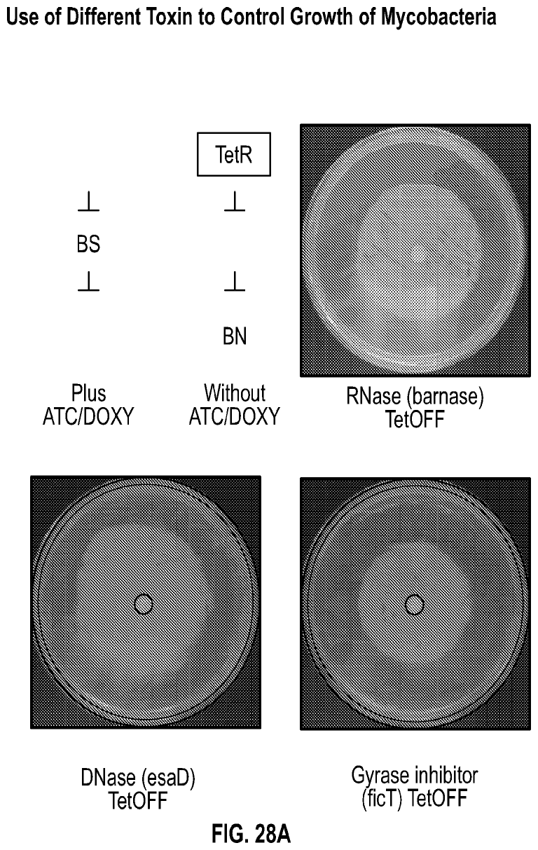

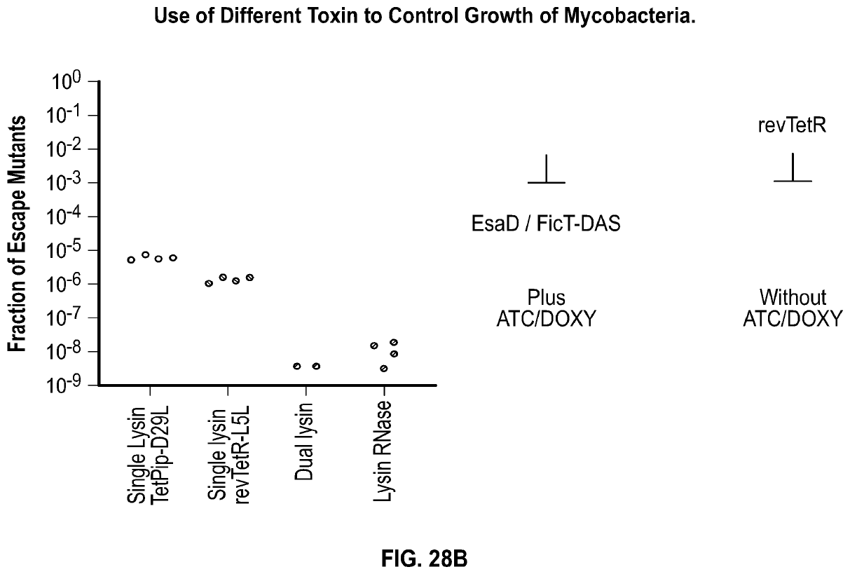

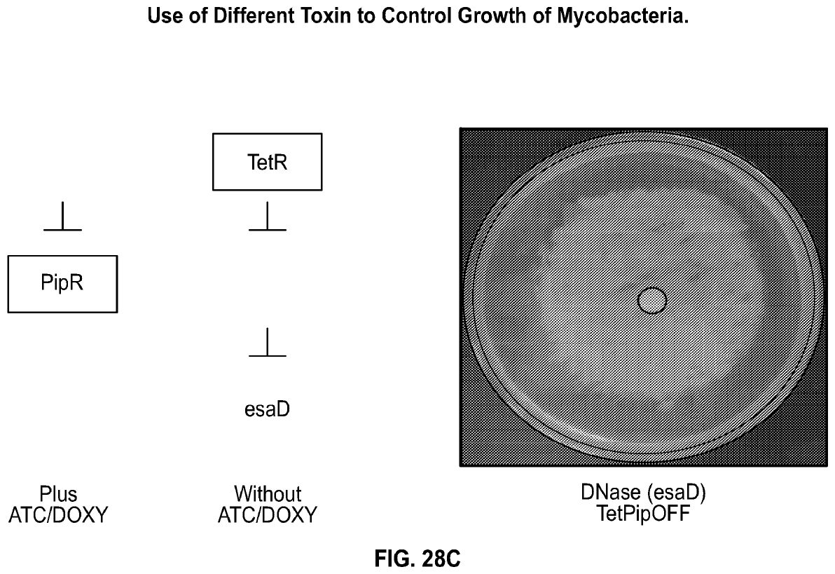

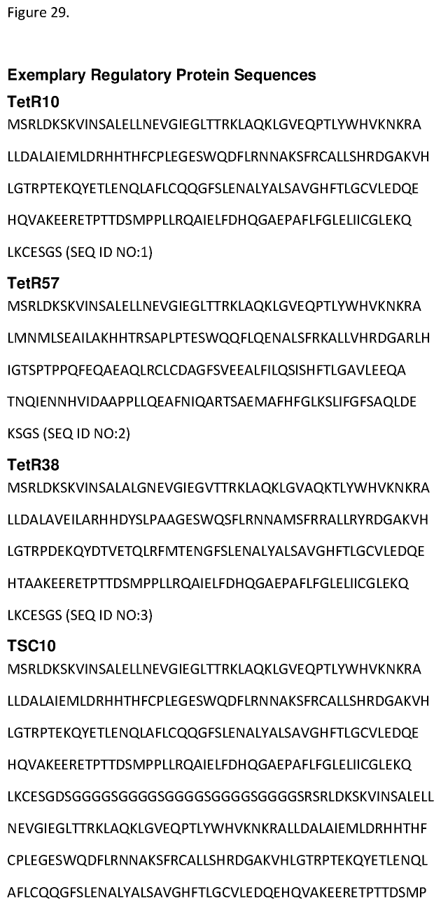

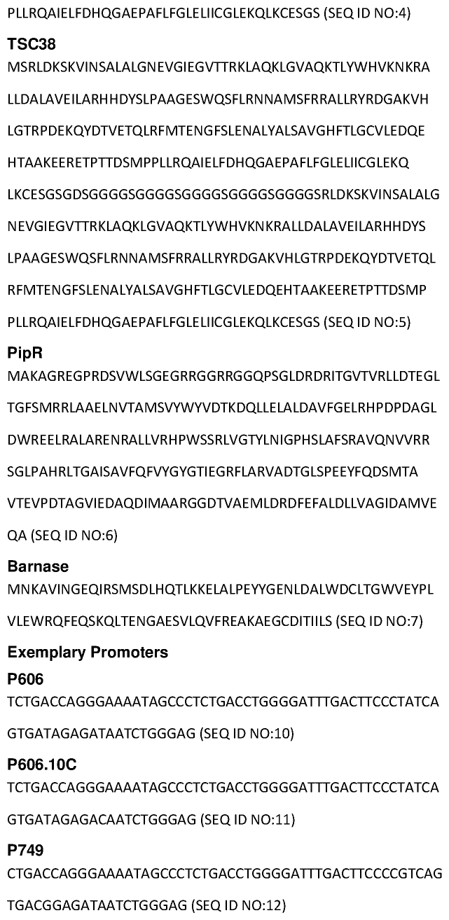

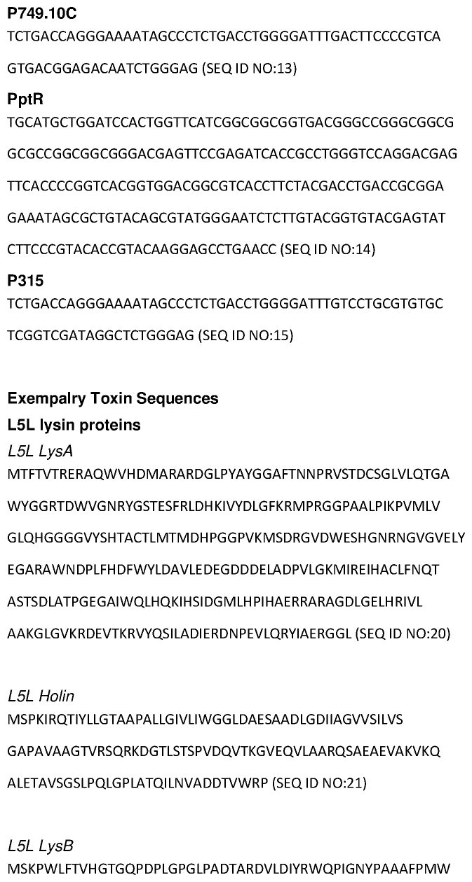

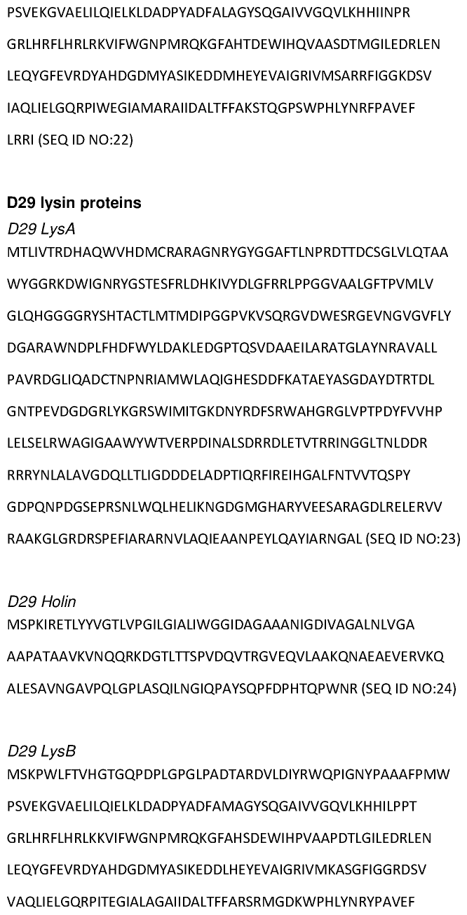

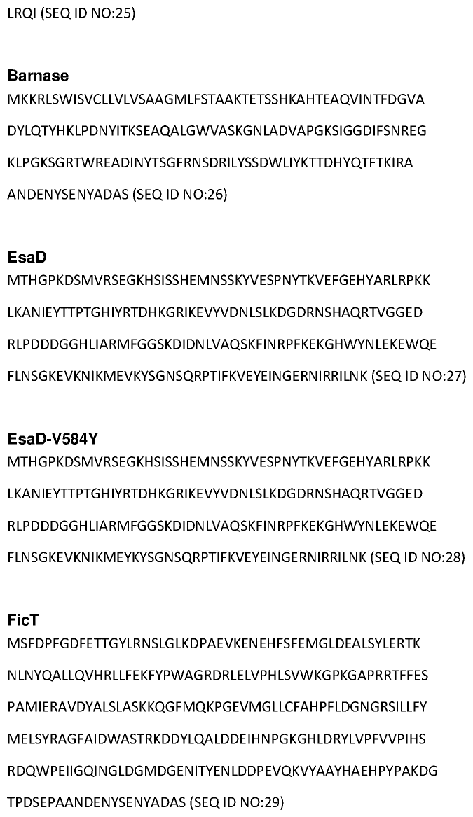

(1) Grow the strain in the presence of ATC (1000 ng/ml) to log phase (OD₅₈₀=0.6-1.0)
(2) Wash cells at least 2 times to remove ATC with PBS by centrifuging at 4000 rpm for 10 min
(3) Resuspend the cells in 7H9 without ATC after the last wash and measure OD₅₈₀
(4) Dilute the cells to OD₅₈₀=0.01 into 10 ml 7H9 with and without ATC and measure OD₅₈₀ and CFUs at indicated time point in the FIG.
(5) Culture the bacteria on agar plates containing ATC (1000 ng/ml)
   Quantification of bacterial CFUs (left) and culture optical density (right) of BCG TetOFF dual lysin strain in 7H9 medium with and without ATC at indicated time points (FIG. 27). The data are means+/−SD from triplicate cultures and representative of two independent experiments. Error bars for OD₅₈₀ values are too small to be depicted.

### EXAMPLE 4

After bladder cancer resection, a patient, e.g., with non-muscle invasive bladder cancer, is intravesicularly treated with a BCG TetOFF or BCG TetOn strain, e.g., for induction and maintenance immunotherapy. For immunotherapy, in one embodiment, up to 10e9 CFU BCG TetOFF or TetON is suspended in about 50 cc of saline and administered using a catheter. Patients are instructed to lie on the abdomen for 15 minutes and retain the suspension for up to 2 hours. At the same time, percutaneous BCG TetOFF or TetON (in one embodiment, approximately 10e7 CFUs) is administered using a sterile 28 gauge needle. Intravesical and percutaneous treatments are repeated each week for 6 weeks. For TetOFF strains sensitive to regulation with doxycycline, doxycycline may be administered for about 6 weeks of treatment. In the case of complications, such as BCGosis, doxycycline treatment is aborted (for doxycycline-addicted BCG strains) or initiated (for strains in which the kill-switches are induced with doxycycline). Efficacy of all recombinant BCG strains is likely similar to that of WT BCG, which has been reported to increase the median recurrence-free survival from 35.7 months (95% confidence interval 25.1 to 56.8) to 76.8 months (64.3 to 93.2, log rank p<0.0001).

### EXAMPLE 5

Biotin (aka vitamin H or vitamin B7) is a cofactor required by several enzymes including several involved in fatty acid biosynthesis. Mycobacteria such as Mtb and M. bovis BCG are able to synthesize biotin. Mutants of Mtb that are unable to synthesize this cofactor, so-called biotin auxotrophs, can grow in vitro, if a sufficient concentration of biotin is added to the growth medium, but get killed in vivo because they are unable to scavenge biotin from the host (Tiwari et al., 2018; Woong et al., 2011). Due to the strong attenuation caused by mutations that inactivate biotin synthesis in Mtb, using biotin auxotrophic mutants is thus a strategy to increase safety of bacterial vaccines such as BCG. And because biotin is an essential cofactor for other bacterial pathogens, such as Francisella tularensis, Acinetobacter baumannii, Klebsiella pneumoniae, and Pseudomonas aeruginosa, this approach may have wide applications.

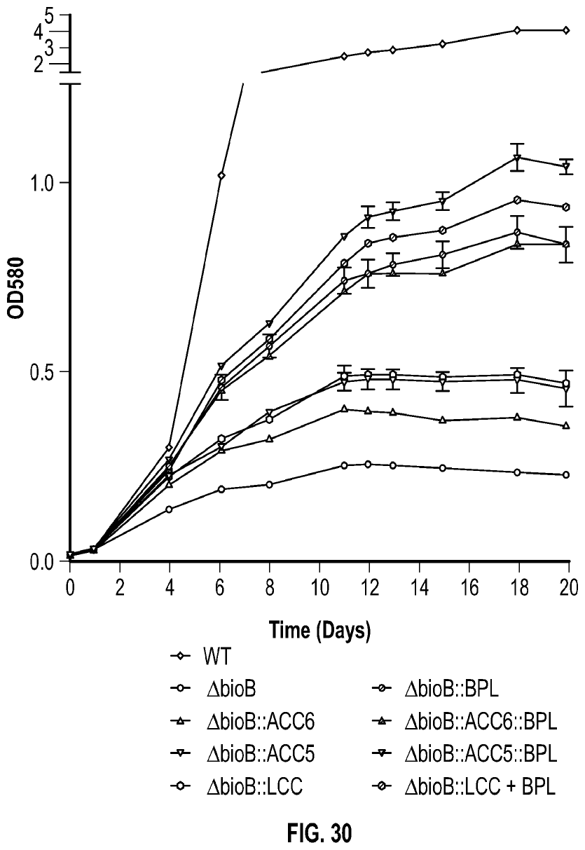

A drawback of bacterial mutants that are auxotrophic for an essential molecule is that their viability cannot be controlled during an infection as is possible with the TetOFF and TetOn, e.g., doxycycline-regulated, kill switches. Too fast of a clearance is likely to reduce or prevent the efficacy of BCG and other bacterial vaccines. Lipoic acid and biotin are unusual cofactors because both need to be covalently attached to the enzymes that require them for activity. This feature can be exploited to control the period of time an auxotroph is viable after transfer into a cofactor-free environment. Both biotin-dependent enzymes and the biotin-protein ligase (BPL), which attaches biotin to all biotin-dependent enzymes, were overexpressed. As shown in FIG. 30, overexpression of BPL and the enzymes ACC6, ACC5, or LCC, increased growth of the biotin auxotroph Mtb ΔbioB (in which the biotin synthase, BioB, has been inactivated). A similar approach allows for control of the viability of bacteria that are auxotrophic for lipoic acid. Other systems that may be employed include overexpression of a small-soluble protein, such as GFP, that contains the so-called AviTag™ (https://www.genecopocia.com/tech/avitag-biotinylation-tag/), as the AviTag™ becomes biotinylated after coexpression of the biotin-protein ligase of E. coli, and expression of a protein that binds biotin non-covalently, such as streptavidin or tamavidin.

### EXAMPLE 6

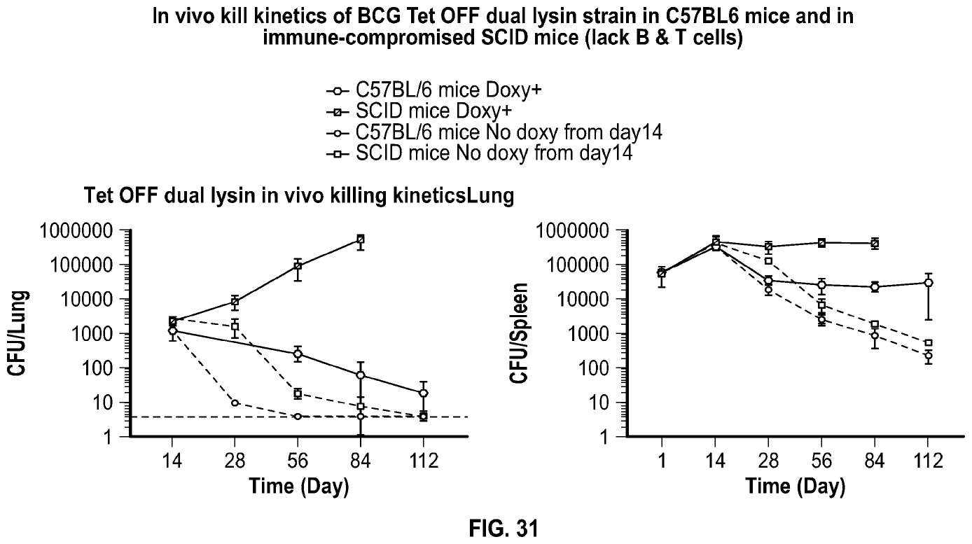

FIG. 31 shows that the BCG Tet OFF dual lysin strain is safe even in immune compromised SCID mice.

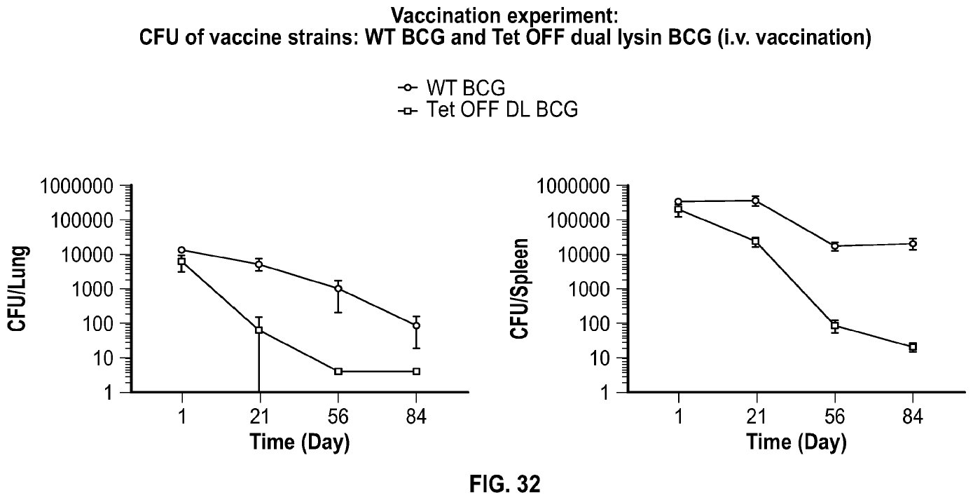

FIG. 32 illustrates CFU in the lung and spleen after wild type or BCG Tet OFF dual lysin strain i.v. administration.

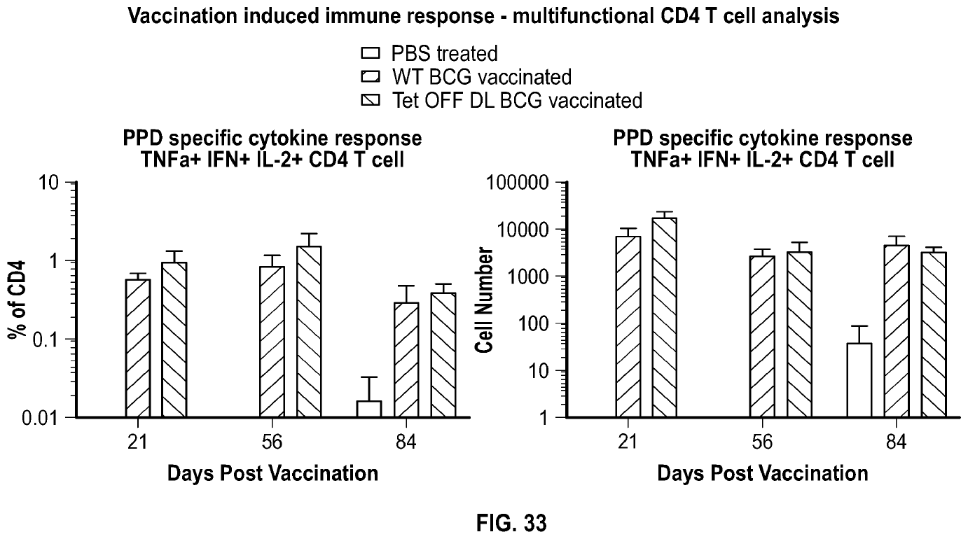

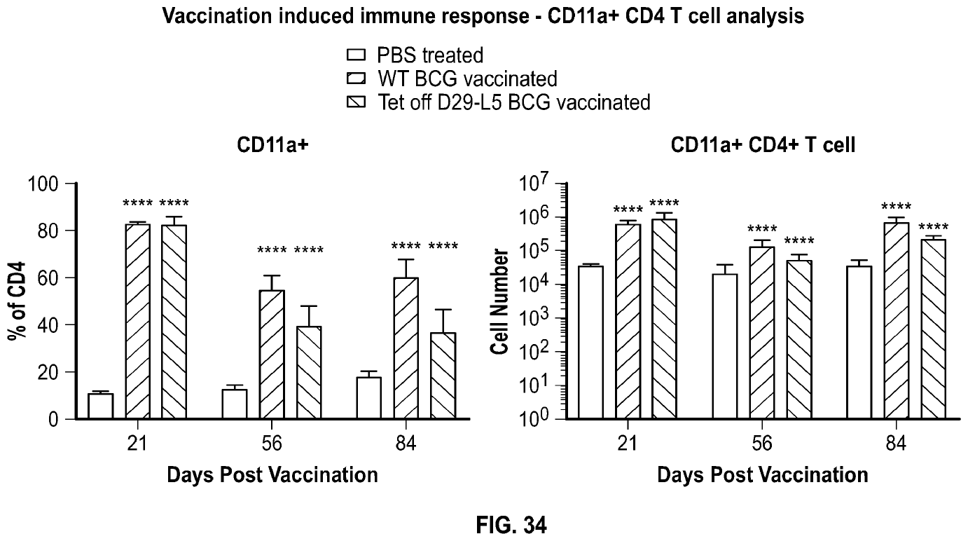

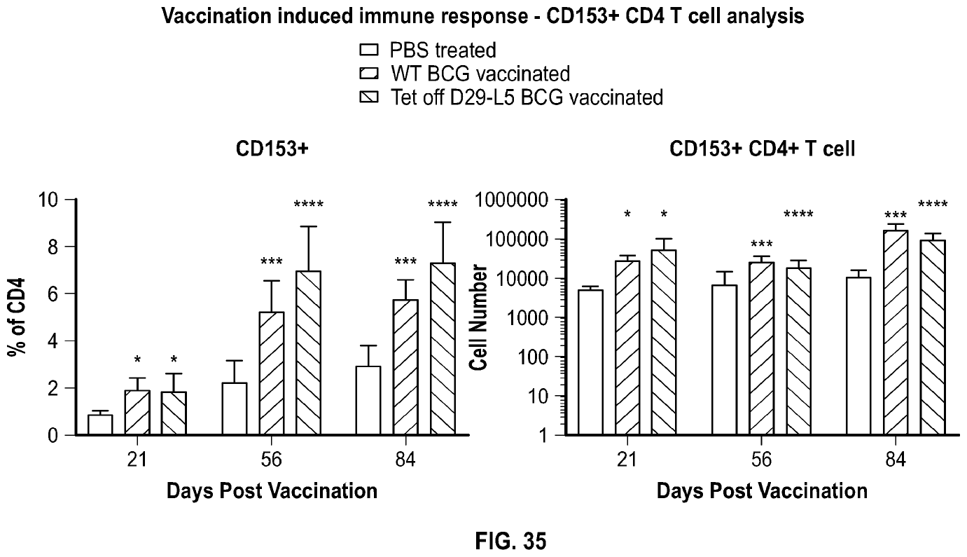

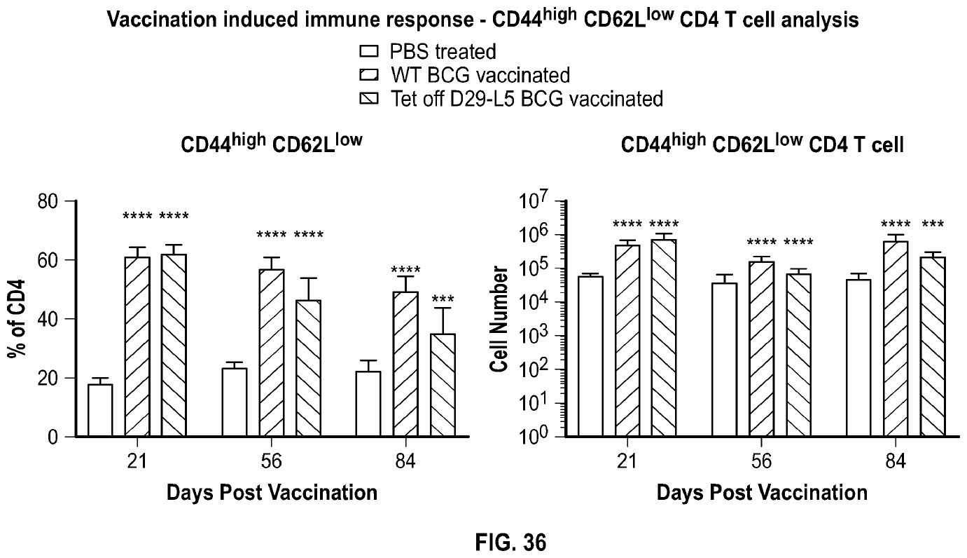

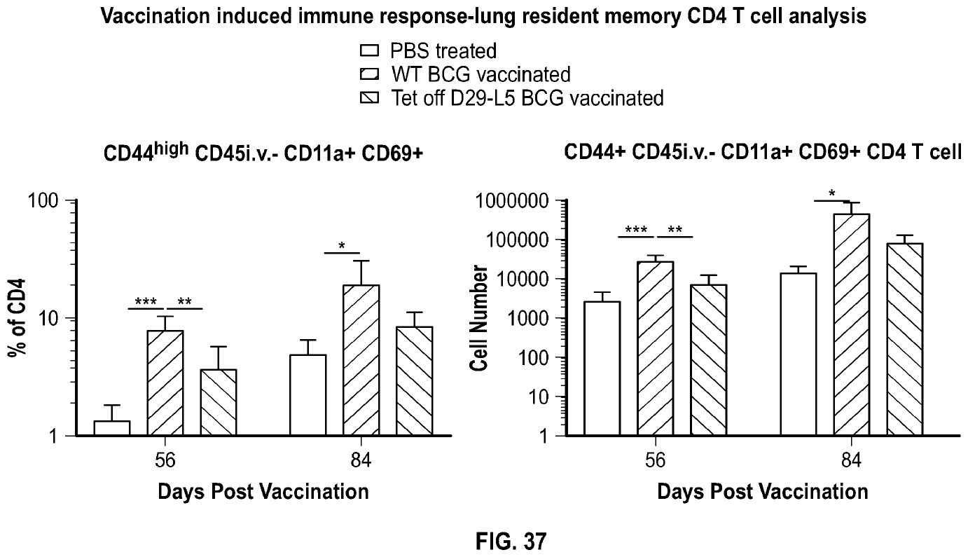

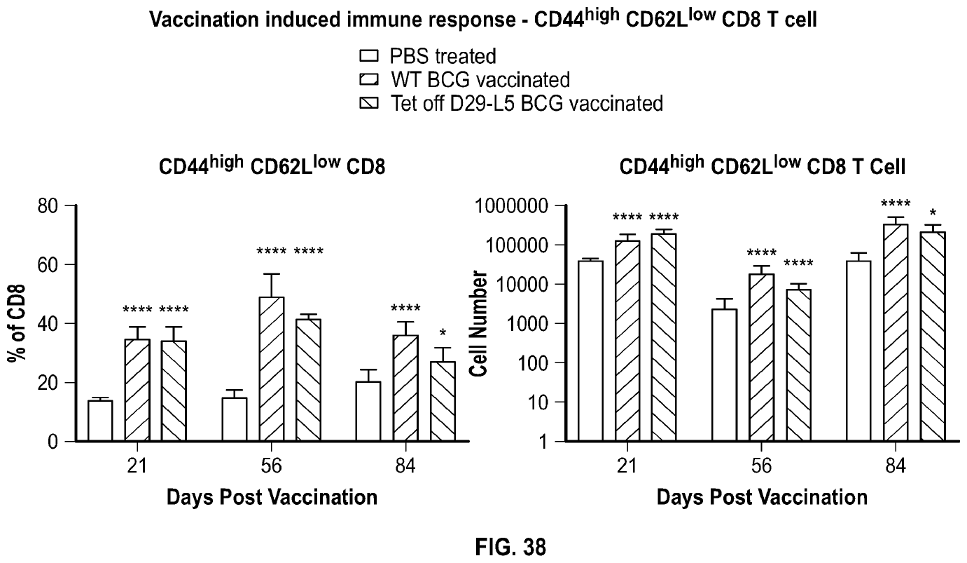

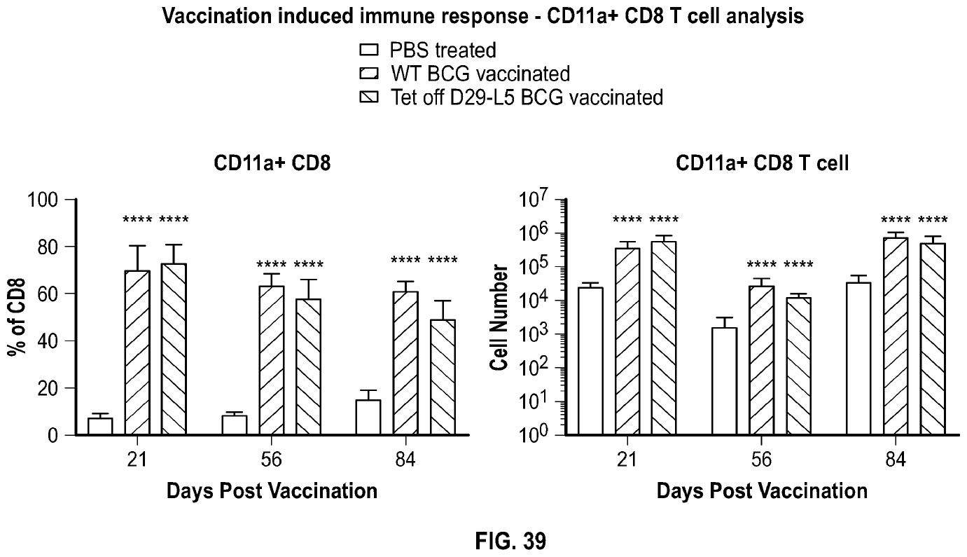

FIGS. 33-39 show cell analyses of the vaccination induced immune response.

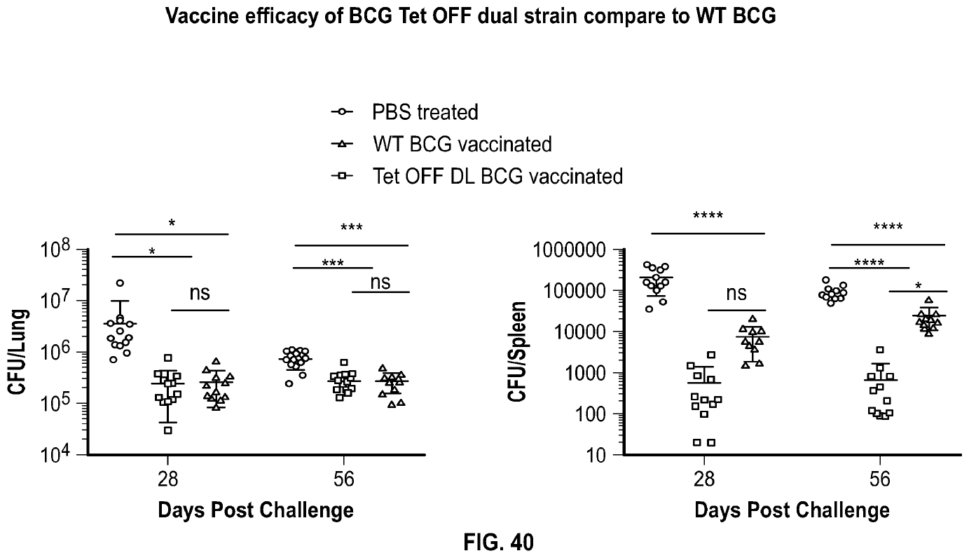

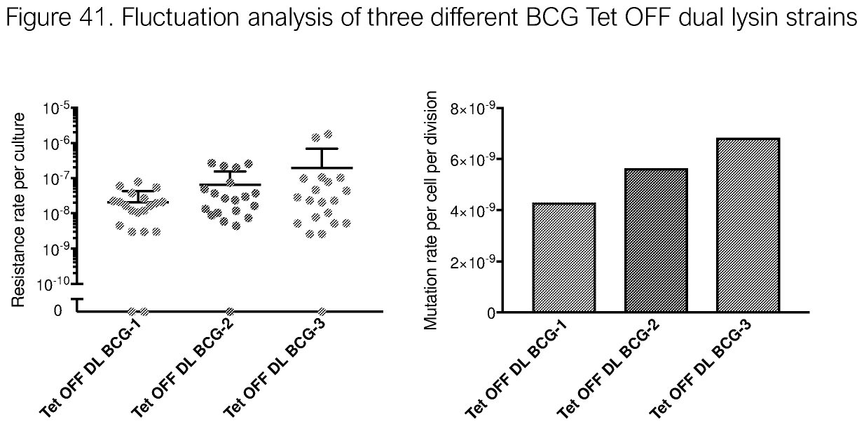

FIG. 40 illustrates the vaccine efficacy of BCG Tet OFF dual strain compared to WT BCG. The BCG Tet OFF dual strain protects as well as WT BCG in the lung.

Thus, the BCG Tet OFF dual lysin strain elicits immune responses that are similar to those elicited by WT BCG and the protective efficacy of the BCG Tet OFF dual lysin strain is similar to that of wild type BCG.

### REFERENCES

Alexandroff et al., Lancet 353:1689 (1999).
Altschul et al., J. Mol. Biol., 215:403 (1990).
Altschul et al., Nucleic Acids Res., 25:3389 (1997).
Andersen & Doherty, Nat. Rev. Microbiol., 3:656 (2005).
Babjuk, M. et al., Eur. Urol., 59:997 (2011).
Boldrin et al., Nucleic Acids Res., 38:e134 (2010).
Casanova & Abel, Annu. Rev. Immunol., 20:581 (2002).
Darrah et al., Nature, 577:95 (2020).
Gish & States, Nature Genet., 3:266 (1993).
Gontero et al., Eur. Urol., 57:410 (2010).
Jemal, A. et al, CA Cancer J. Clin., 61:69 (2011).
Klotzsche et al., Nucleic Acids Res., 37:1778 (2009).
Madden et al., Meth. Enzymol., 266:131 (1996).
Pearson, Methods Enzymol., 183:63 (1990).
Perez-Jacoiste et al., Medicine (Baltimore), 93:236 (2014).
Sallin et al., Nat. Microbio., 3:1198 (2018).
Schnappinger & Ehrt, Microbiol. Spectr., 2:pii 03 (2014).
Tiwari et al., Sci. Transl. Med., 10:pii:eaa11803 (2018).
Woong et at, PLoS Pathog., 7: e1002264 (2011)
Zbar et al., J. Natl. Cancer Inst., 46:831 (1971).
Zhang & Madden, Genome Res., 7:649 (1997).

All publications, patents and patent applications are incorporated herein by reference. While in the foregoing specification, this invention has been described in relation to certain embodiments thereof, and many details have been set forth for purposes of illustration, it will be apparent to those skilled in the art that the invention is susceptible to additional embodiments and that certain of the details herein may be varied considerably without departing from the basic principles of the invention.

1. An isolated recombinant cell comprising a vector comprising at least two expression cassettes, wherein a first expression cassette comprises a first transcriptional regulatory region that includes a promoter operably linked to a first open reading frame encoding a first bacteriocidal gene product, wherein transcription from the first transcriptional regulatory region is controlled in trans by a first transcriptional regulatory protein, wherein a second expression cassette comprises a second transcriptional regulatory region that includes a promoter operably linked to a second open reading frame encoding a second bacteriocidal gene product, wherein transcription from the second transcriptional regulatory region is controlled in trans by a second transcriptional regulatory protein, wherein the first and second open reading frames encode different bacteriocidal gene products, wherein the activity of at least the first transcriptional regulatory protein is controlled by a first exogenous agent, and wherein optionally the vector further comprises a third expression cassette comprising a third transcriptional regulatory region that includes a promoter operably linked to a third open reading frame encoding the second transcriptional regulatory protein, wherein transcription from the third transcriptional regulatory region is controlled in trans by a third transcriptional regulatory protein, wherein the activity of the third transcriptional regulatory protein is controlled by the exogenous agent.

2. The isolated recombinant cell of claim 1 wherein the first and the second transcriptional regulatory regions are controlled by the same transcriptional regulatory protein.

3. The isolated recombinant cell of claim 2 wherein the activity of the second transcriptional regulatory region is controlled by the first exogenous agent.

4. The isolated recombinant cell of claim 2 wherein the activity of the second transcriptional regulatory region is controlled by a second exogenous agent that is different than the first exogenous agent.

5. The isolated recombinant cell of claim 1 wherein the exogenous agent comprises a tetracycline or a macrolide.

6. The isolated recombinant cell of claim 1 wherein the presence of the exogenous agent induces expression of the first and the second bacteriocidal gene products from the vector.

7. The isolated recombinant cell of claim 1 wherein the first and the second transcriptional regulatory regions are controlled by different transcriptional regulatory proteins.

8. The isolated recombinant cell of claim 7 comprising the third expression cassette.

9-12. (canceled)

13. The isolated recombinant cell of claim 1 wherein the first transcriptional regulatory protein or the second transcriptional regulatory protein comprise TetR, Lad or AraC.

14. The isolated recombinant cell of claim 1 wherein the first open reading frame or the second open reading frame encode a lysin or a nuclease.

15. The isolated recombinant cell of claim 1 wherein the first open reading frame arid the second open reading frame encode different lysins or different nucleases.

16-22. (canceled)

23. The isolated recombinant cell of claim 1 which further comprises one or more genes of interest.

24. (canceled)

25. The isolated recombinant cell of claim 1 which is a Mycobacteria.

26-29. (canceled)

30. The isolated recombinant cell of claim 1 wherein the first bacteriocidal gene product or the second bacteriocidal gene product is a heterologous bacteriocidal gene product.

31. The isolated recombinant cell of claim 1 wherein the first bacteriocidal gene product or the second bacteriocidal gene product is a phage gene product.

32-43. (canceled)

44. A method to immunize a mammal against Mycobacterium infection, to treat Mycobacterium infection in as mammal or inhibit or treat bladder cancer in a mammal, comprising: administering to the mammal an effective amount of the recombinant cell of claim 1 and administering an amount of the exogenous agent effective to control viability of the recombinant cell.

45. The method of claim 44 wherein the administration of the exogenous agent increases expression of the first and the second bacteriocidal gene products.

46. The method of claim 44 wherein the exogenous agent is administered in an amount or for a period of time effective to lyse the recombinant cell or maintain the viability of the recombinant cell.

47. The method of claim 44 wherein the administration of the exogenous agent represses expression of the first and the second bacteriocidal gene products.

48. (canceled)

49. The method of claim 47 further comprising decreasing the amount of exogenous agent administered so that the first and the second bacteriocidal gene products are expressed.

50-59. (canceled)


SEQUENCE LISTING (29 sequences)

SEQ ID NO: 1 - protein, 207 aa, Artificial Sequence
  A synthetic sequence
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 2 - protein, 204 aa, Artificial Sequence
  A synthetic sequence
MSRLDKSKVINSALELLNEVGIEGLTTRKLAQKLGVEQPTLYWHVKNKRALMNMLSEAILAKHHTRSAPLPTESWQQFLQENALSFRKALLVHRDGARLHIGTSPTPPQFEQAEAQLRCLCDAGFSVEEALFILQSISHFTLGAVLEEQATNQIENNHVIDAAPPLLQEAFNIQARTSAEMAFHFGLKSLIFGFSAQLDEKSGS

SEQ ID NO: 3 - protein, 207 aa, Artificial Sequence
  A synthetic sequence
MSRLDKSKVINSALALGNEVGIEGVTTRKLAQKLGVAQKTLYWHVKNKRALLDALAVEILARHHDYSLPAAGESWQSFLRNNAMSFRRALLRYRDGAKVHLGTRPDEKQYDTVETQLRFMTENGFSLENALYALSAVGHFTLGCVLEDQEHTAAKEERETPTTDSMPPLLRQAIELFDHQGAEPAFLFGLELIICGLEKQLKCESGS

SEQ ID NO: 4 - protein, 440 aa, Artificial Sequence
  A synthetic sequence
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGDSGGGGSGGGGSGGGGSGGGGSGGGGSRSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 5 - protein, 440 aa, Artificial Sequence
  A synthetic sequence
MSRLDKSKVINSALALGNEVGIEGVTTRKLAQKLGVAQKTLYWHVKNKRALLDALAVEILARHHDYSLPAAGESWQSFLRNNAMSFRRALLRYRDGAKVHLGTRPDEKQYDTVETQLRFMTENGFSLENALYALSAVGHFTLGCVLEDQEHTAAKEERETPTTDSMPPLLRQAIELFDHQGAEPAFLFGLELIICGLEKQLKCESGSGDSGGGGSGGGGSGGGGSGGGGSGGGGSRLDKSKVINSALALGNEVGIEGVTTRKLAQKLGVAQKTLYWHVKNKRALLDALAVEILARHHDYSLPAAGESWQSFLRNNAMSFRRALLRYRDGAKVHLGTRPDEKQYDTVETQLRFMTENGFSLENALYALSAVGHFTLGCVLEDQEHTAAKEERETPTTDSMPPLLRQAIELFDHQGAEPAFLFGLELIICGLEKQLKCESGS

SEQ ID NO: 6 - protein, 252 aa, Artificial Sequence
  A synthetic sequence
MAKAGREGPRDSVWLSGEGRRGGRRGGQPSGLDRDRITGVTVRLLDTEGLTGFSMRRLAAELNVTAMSVYWYVDTKDQLLELALDAVFGELRHPDPDAGLDWREELRALARENRALLVRHPWSSRLVGTYLNIGPHSLAFSRAVQNVVRRSGLPAHRLTGAISAVFQFVYGYGTIEGRFLARVADTGLSPEEYFQDSMTAVTEVPDTAGVIEDAQDIMAARGGDTVAEMLDRDFEFALDLLVAGIDAMVEQA

SEQ ID NO: 7 - protein, 90 aa, Artificial Sequence
  A synthetic sequence
MNKAVINGEQIRSMSDLHQTLKKELALPEYYGENLDALWDCLTGWVEYPLVLEWRQFEQSKQLTENGAESVLQVFREAKAEGCDITIILS

SEQ ID NO: 8-9 - skipped (2 entries)

SEQ ID NO: 10 - DNA, 71 bp, Artificial Sequence
  A synthetic sequence
tctgaccagggaaaatagccctctgacctggggatttgacttccctatcagtgatagagataatctgggag

SEQ ID NO: 11 - DNA, 71 bp, Artificial Sequence
  A synthetic sequence
tctgaccagggaaaatagccctctgacctggggatttgacttccctatcagtgatagagacaatctgggag

SEQ ID NO: 12 - DNA, 70 bp, Artificial Sequence
  A synthetic sequence
ctgaccagggaaaatagccctctgacctggggatttgacttccccgtcagtgacggagataatctgggag

SEQ ID NO: 13 - DNA, 71 bp, Artificial Sequence
  A synthetic sequence
tctgaccagggaaaatagccctctgacctggggatttgacttccccgtcagtgacggagacaatctgggag

SEQ ID NO: 14 - DNA, 231 bp, Artificial Sequence
  A synthetic sequence
tgcatgctggatccactggttcatcggcggcggtgacgggccgggcggcggcgccggcggcgggacgagttccgagatcaccgcctgggtccaggacgagttcaccccggtcacggtggacggcgtcaccttctacgacctgaccgcggagaaatagcgctgtacagcgtatgggaatctcttgtacggtgtacgagtatcttcccgtacaccgtacaaggagcctgaacc

SEQ ID NO: 15 - DNA, 71 bp, Artificial Sequence
  A synthetic sequence
tctgaccagggaaaatagccctctgacctggggatttgtcctgcgtgtgctcggtcgataggctctgggag

SEQ ID NO: 16-19 - skipped (4 entries)

SEQ ID NO: 20 - protein, 292 aa, Artificial Sequence
  A synthetic sequence
MTFTVTRERAQWVHDMARARDGLPYAYGGAFTNNPRVSTDCSGLVLQTGAWYGGRTDWVGNRYGSTESFRLDHKIVYDLGFKRMPRGGPAALPIKPVMLVGLQHGGGGVYSHTACTLMTMDHPGGPVKMSDRGVDWESHGNRNGVGVELYEGARAWNDPLFHDFWYLDAVLEDEGDDDELADPVLGKMIREIHACLFNQTASTSDLATPGEGAIWQLHQKIHSIDGMLHPIHAERRARAGDLGELHRIVLAAKGLGVKRDEVTKRVYQSILADIERDNPEVLQRYIAERGGL

SEQ ID NO: 21 - protein, 131 aa, Artificial Sequence
  A synthetic sequence
MSPKIRQTIYLLGTAAPALLGIVLIWGGLDAESAADLGDIIAGVVSILVSGAPAVAAGTVRSQRKDGTLSTSPVDQVTKGVEQVLAARQSAEAEVAKVKQALETAVSGSLPQLGPLATQILNVADDTVWRP

SEQ ID NO: 22 - protein, 254 aa, Artificial Sequence
  A synthetic sequence
MSKPWLFTVHGTGQPDPLGPGLPADTARDVLDIYRWQPIGNYPAAAFPMWPSVEKGVAELILQIELKLDADPYADFALAGYSQGAIVVGQVLKHHIINPRGRLHRFLHRLRKVIFWGNPMRQKGFAHTDEWIHQVAASDTMGILEDRLENLEQYGFEVRDYAHDGDMYASIKEDDMHEYEVAIGRIVMSARRFIGGKDSVIAQLIELGQRPIWEGIAMARAIIDALTFFAKSTQGPSWPHLYNRFPAVEFLRRI

SEQ ID NO: 23 - protein, 493 aa, Artificial Sequence
  A synthetic sequence
MTLIVTRDHAQWVHDMCRARAGNRYGYGGAFTLNPRDTTDCSGLVLQTAAWYGGRKDWIGNRYGSTESFRLDHKIVYDLGFRRLPPGGVAALGFTPVMLVGLQHGGGGRYSHTACTLMTMDIPGGPVKVSQRGVDWESRGEVNGVGVFLYDGARAWNDPLFHDFWYLDAKLEDGPTQSVDAAEILARATGLAYNRAVALLPAVRDGLIQADCTNPNRIAMWLAQIGHESDDFKATAEYASGDAYDTRTDLGNTPEVDGDGRLYKGRSWIMITGKDNYRDFSRWAHGRGLVPTPDYFVVHPLELSELRWAGIGAAWYWTVERPDINALSDRRDLETVTRRINGGLTNLDDRRRRYNLALAVGDQLLTLIGDDDELADPTIQRFIREIHGALFNTVVTQSPYGDPQNPDGSEPRSNLWQLHELIKNGDGMGHARYVEESARAGDLRELERVVRAAKGLGRDRSPEFIARARNVLAQIEAANPEYLQAYIARNGAL

SEQ ID NO: 24 - protein, 141 aa, Artificial Sequence
  A synthetic sequence
MSPKIRETLYYVGTLVPGILGIALIWGGIDAGAAANIGDIVAGALNLVGAAAPATAAVKVNQQRKDGTLTTSPVDQVTRGVEQVLAAKQNAEAEVERVKQALESAVNGAVPQLGPLASQILNGIQPAYSQPFDPHTQPWNR

SEQ ID NO: 25 - protein, 254 aa, Artificial Sequence
  A synthetic sequence
MSKPWLFTVHGTGQPDPLGPGLPADTARDVLDIYRWQPIGNYPAAAFPMWPSVEKGVAELILQIELKLDADPYADFAMAGYSQGAIVVGQVLKHHILPPTGRLHRFLHRLKKVIFWGNPMRQKGFAHSDEWIHPVAAPDTLGILEDRLENLEQYGFEVRDYAHDGDMYASIKEDDLHEYEVAIGRIVMKASGFIGGRDSVVAQLIELGQRPITEGIALAGAIIDALTFFARSRMGDKWPHLYNRYPAVEFLRQI

SEQ ID NO: 26 - protein, 164 aa, Artificial Sequence
  A synthetic sequence
MKKRLSWISVCLLVLVSAAGMLFSTAAKTETSSHKAHTEAQVINTFDGVADYLQTYHKLPDNYITKSEAQALGWVASKGNLADVAPGKSIGGDIFSNREGKLPGKSGRTWREADINYTSGFRNSDRILYSSDWLIYKTTDHYQTFTKIRAANDENYSENYADAS

SEQ ID NO: 27 - protein, 195 aa, Artificial Sequence
  A synthetic sequence
MTHGPKDSMVRSEGKHSISSHEMNSSKYVESPNYTKVEFGEHYARLRPKKLKANIEYTTPTGHIYRTDHKGRIKEVYVDNLSLKDGDRNSHAQRTVGGEDRLPDDDGGHLIARMFGGSKDIDNLVAQSKFINRPFKEKGHWYNLEKEWQEFLNSGKEVKNIKMEVKYSGNSQRPTIFKVEYEINGERNIRRILNK

SEQ ID NO: 28 - protein, 195 aa, Artificial Sequence
  A synthetic sequence
MTHGPKDSMVRSEGKHSISSHEMNSSKYVESPNYTKVEFGEHYARLRPKKLKANIEYTTPTGHIYRTDHKGRIKEVYVDNLSLKDGDRNSHAQRTVGGEDRLPDDDGGHLIARMFGGSKDIDNLVAQSKFINRPFKEKGHWYNLEKEWQEFLNSGKEVKNIKMEYKYSGNSQRPTIFKVEYEINGERNIRRILNK

SEQ ID NO: 29 - protein, 271 aa, Artificial Sequence
  A synthetic sequence
MSFDPFGDFETTGYLRNSLGLKDPAEVKENEHFSFEMGLDEALSYLERTKNLNYQALLQVHRLLFEKFYPWAGRDRLELVPHLSVWKGPKGAPRRTFFESPAMIERAVDYALSLASKKQGFMQKPGEVMGLLCFAHPFLDGNGRSILLFYMELSYRAGFAIDWASTRKDDYLQALDDEIHNPGKGHLDRYLVPFVVPIHSRDQWPEIIGQINGLDGMDGENITYENLDDPEVQKVYAAYHAEHPYPAKDGTPDSEPAANDENYSENYADAS